(19) United States
(12) Patent Application Publication
(10) Pub. No.: US 2019/0375692 A1
(43) Pub. Date: Dec. 12, 2019

(54) CARBON NANOBELT AND PRODUCTION METHOD THEREFOR

(71) Applicant: NATIONAL UNIVERSITY CORPORATION NAGOYA UNIVERSITY, Nagoya-shi, Aichi (JP)

(72) Inventors: Kenichiro ITAMI, Nagoya-shi, Aichi (JP); Yasutomo SEGAWA, Nagoya-shi, Aichi (JP); Guillaume POVIE, Nagoya-shi, Aichi (JP); Yuhei MIYAUCHI, Nagoya-shi, Aichi (JP); Taishi NISHIHARA, Nagoya-shi, Aichi (JP)

(21) Appl. No.: 16/331,867

(22) PCT Filed: Feb. 14, 2018
(86) PCT No.: PCT/JP2018/005077

§ 371 Date: Mar. 8, 2019

(30) Foreign Application Priority Data
Feb. 14, 2017 (JP) 2017-025127

(51) Int. Cl. C07C 1/28; B01J 23/755; C07C 15/20; C07C 17/263; C07C 25/24; C07C 47/55; C07C 43/225

(52) U.S. Cl. CPC C07C 1/28; B01J 23/755; C07C 15/20; C07C 2523/755; C07C 25/24; C07C 47/55; C07C 43/225; C07C 17/2637

(57) ABSTRACT

A carbon nanobelt represented by formula (1) was synthesized by chemical synthesis:

[symbol]

wherein each Ar is identical or different and represents an aromatic hydrocarbon ring; each R¹ is identical or different and represents hydrogen, alkyl, aryl, or alkoxy; and n represents an integer of 0 or more.

### TECHNICAL FIELD

The present invention relates to carbon nanobelts and production methods for them.

### BACKGROUND ART

Cyclic aromatic compounds that serve as a partial framework of a carbon nanotube (CNT) are a group of promising molecules expected to exhibit various types of functional expression and to have a variety of applications, such as their specific electronic properties due to n-conjugation, inclusion of a guest molecule through a hole inside the compound, and the use of the compound in synthesis of CNT (e.g., see NPL 1). In particular, cyclic aromatic compounds have recently drawn attention as a template molecule for bottom-up synthesis of CNT, which will enable the construction of uniformly structured CNT, which has been difficult to create by traditional methods (e.g., see NPL 2). A study reports that CNT can be synthesized by a CVD method, using a cycloparaphenylene compound (CPP) having benzene rings cyclically attached at their para-position as a template molecule.

However, cyclic aromatic compounds synthesized thus far, including CPP, are formed such that the target ring structure is cyclically single bonded; thus, cleavage of a single carbon-carbon bond easily opens the cyclic structure. A group of molecules that have aromatic rings curved like a belt, and that require cleavage of at least two carbon-carbon bonds to open the ring structure, are defined as carbon nanobelts (e.g., see NPL 3). A carbon nanobelt is the smallest unit of CNT that can be illustrated as a molecule, and is expected to be more rigid than conventional cyclic aromatic compounds, showing promise as an excellent template molecule in characteristic evaluation of CNT and CNT synthesis. However, the synthesis of a carbon nanobelt has been unsuccessful.

### CITATION LIST

### Non-Patent Literature

NPL 1: Nat. Rev. Mater., 2016, 1, 15002
NPL 2: Angew. Chem. Int. Ed., 2016, 55, 5136
NPL 3: Org. Lett., 2016, 18, 1430

### SUMMARY OF INVENTION

### Technical Problem

An object of the present invention is to synthesize a carbon nanobelt by chemical synthesis in light of conventional techniques.

### Solution to Problem

The present inventors conducted extensive research to solve the problem, and found that a target carbon nanobelt can be synthesized by designing and synthesizing a specific cyclic compound as a synthetic intermediate, and then performing an intermolecular coupling reaction. The inventors also found that a carbon nanobelt having a desired structure according to the present invention emits red fluorescence. On the basis of these findings, the inventors conducted further research and completed the present invention. Specifically, the present invention includes the following subject matter.

Item 1. A carbon nanobelt represented by formula (1):
wherein each Ar is identical or different and represents an aromatic hydrocarbon ring; each R¹ is identical or different and represents hydrogen, alkyl, aryl, or alkoxy; and n represents an integer of 0 or more.
Item 2. The carbon nanobelt according to Item 1 represented by formula (1A):
wherein Ar and n are as defined above.
Item 3. The carbon nanobelt according to Item 1 or 2, wherein Ar in formula (1) or formula (1A) represents a benzene ring or a naphthalene ring.
Item 4. The carbon nanobelt according to any one of Items 1 to 3, wherein n in formula (1) or formula (1A) represents an integer of 0 to 10.
Item 5. A method for producing the carbon nanobelt of any one of Items 1 to 4, the method comprising reacting, in the presence of a nickel catalyst, a cyclic compound represented by formula (2):
wherein R¹ and n are as defined above; and each Ar′ is identical or different and represents an aromatic hydrocarbon ring having two halogen atoms as substituents.
Item 6. The method according to Item 5, wherein Ar′ in formula (2) is a group represented by formula (5A) or (5B):

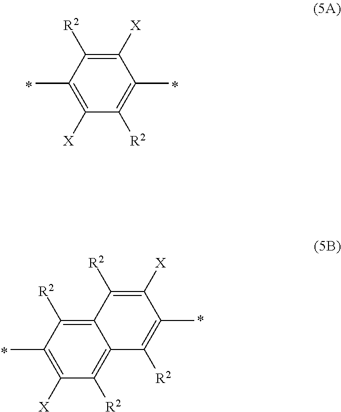

wherein each R² is identical or different and represents hydrogen, alkyl, aryl, or alkoxy; each X is identical or different and represents a halogen atom; and the asterisk “*” represents a binding site.
Item 7. The method according to Item 5 or 6, wherein the cyclic compound represented by formula (2) is obtained by reacting, in the presence of a base, a compound represented by formula (3A) or a salt thereof:

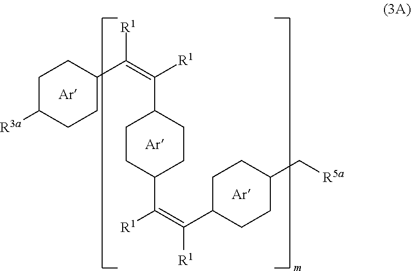

wherein Ar′ and R¹ are as defined above, R^{5a} represents triaryl phosphine or triaryl phosphite, m represents an integer of 0 or more, and R^{3a} represents acyl or a group represented by formula (4A), with the proviso that when m is 0, R^{3a} represents the group represented by formula (4A):

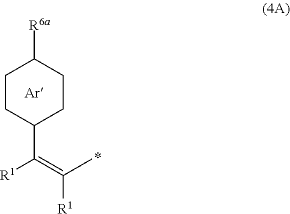

wherein Ar′ and R¹ are as defined above, and R^{6a} represents acyl.
Item 8. A cyclic compound represented by formula (2):
wherein each Ar′ is identical or different and represents an aromatic hydrocarbon ring having two halogen atoms as substituents; each R¹ is identical or different and represents hydrogen, alkyl, aryl, or alkoxy; and n represents an integer of 0 or more.
Item 9. The cyclic compound according to Item 8, wherein Ar′ in formula (2) is a group represented by formula (5A) or (5B):

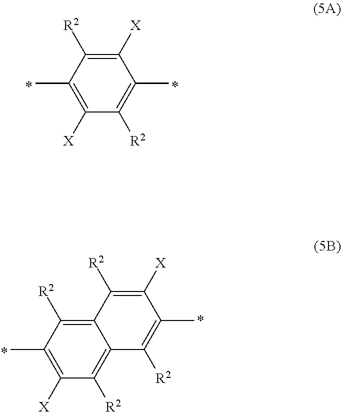

wherein each R² is identical or different and represents hydrogen, alkyl, aryl, or alkoxy; each X is identical or different and represents a halogen atom; and the asterisk “*” represents a binding site.
Item 10. A method for producing the cyclic compound of Item 8 or 9, the method comprising reacting, in the presence of a base, a compound represented by formula (3A) or a salt thereof:

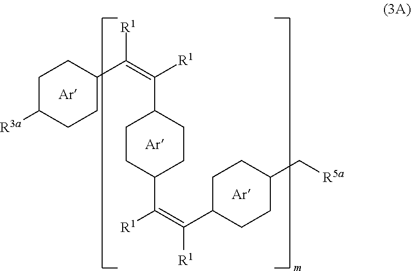

wherein Ar′ and R¹ are as defined above, R^{5a} represents triaryl phosphine or triaryl phosphite, m represents an integer of 0 or more, and R^{3a} represents acyl or a group represented by formula (4A), with the proviso that when m is 0, R^{3a} is the group represented by formula (4):

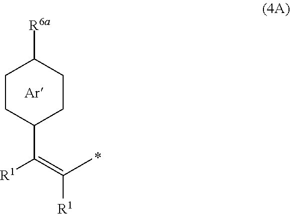

wherein Ar′ and R¹ are as defined above, and R^{6a} represents acyl.
Item 11. A compound represented by formula (3) or a salt thereof:

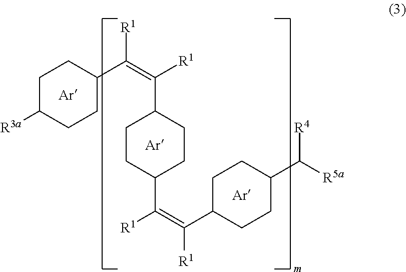

wherein each Ar′ is identical or different and represents an aromatic hydrocarbon ring having two halogen atoms as substituents; each R¹ is identical or different and represents hydrogen, alkyl, aryl, or alkoxy; R⁴ represents a hydrogen atom or a halogen atom; R⁵ represents a halogen atom, triaryl phosphine, or triaryl phosphite; m represents an integer of 0 or more; and R³ represents acyl, dialkoxymethyl, or a group represented by formula (4), with the proviso that when m is 0, R³ is the group represented by formula (4):

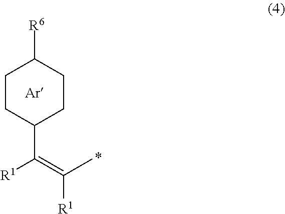

wherein Ar′ and R¹ are as defined above, and R⁶ represents acyl or dialkoxymethyl.

### Advantageous Effects of Invention

The present invention enables the synthesis of a carbon nanobelt.

### BRIEF DESCRIPTION OF DRAWINGS

FIG. 1 illustrates the structure of a cyclic compound 10.(tol)₂ determined by X-ray structure diffraction. The structure, which was drawn with Oak Ridge Thermal Ellipsoid Plot (ORTEP) software, shows a 50% existence probability. To avoid complexity, no hydrogen atoms or solvent molecules are shown.

FIG. 2 illustrates the structure of [12]ICP (carbon nanobelt 11.(CHCl₃)₃) determined by X-ray structure diffraction. The structure, which was drawn with Oak Ridge Thermal Ellipsoid Plot software (ORTEP), shows a 50% existence probability. To avoid complexity, no hydrogen atoms or solvent molecules are shown.

FIG. 3 illustrates an absorption spectrum (solid line) and a fluorescence spectrum (dashed line) of [12]ICP.

FIG. 4 illustrates fluorescence spectra of a single crystal (solid line) and a solution (dashed line) of [12]ICP.

FIG. 5 illustrates fluorescence decay of a single crystal (blue line) and a solution (red line) of [12]ICP.

FIG. 6 illustrates a Raman spectrum of a single crystal of [12]ICP (black line) and a simulated spectrum (gray line) determined by scaling with a theoretical value at B3LYP/6-31G(d) level of 0.9613. Charts b and c are magnified views of chart a.

FIG. 7 illustrates vibrational modes of the Raman peak of [12]ICP. The arrows indicate a force constant.

### DESCRIPTION OF EMBODIMENTS

### 1. Carbon Nanobelt

The carbon nanobelt according to the present invention is a compound represented by formula (1):

wherein each Ar is identical or different and represents an aromatic hydrocarbon ring; each R¹ is identical or different and represents hydrogen, alkyl, aryl, or alkoxy; and n represents an integer of 0 or more.

This carbon nanobelt is a compound having a ring structure composed of repeating units represented by formula (6):

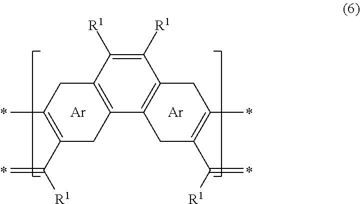

wherein Ar and R¹ are as defined above, and the asterisk “*” represents a binding site.

Specifically, the carbon nanobelt according to the present invention has a belt-like structure in which aromatic hydrocarbon rings optionally having a specific substituent are bound to each other, and at least two carbon-carbon bonds must be cleaved to open the cyclic structure. Thus, the carbon nanobelt according to the present invention is a compound that is more rigid and stable than conventional cyclic aromatic compounds.

Alkyl represented by R¹ in formula (1) includes linear or branched C₁₋₆ alkyl such as methyl, ethyl, n-propyl, isopropyl, n-butyl, isobutyl, sec-butyl, tert-butyl, n-pentyl, and n-hexyl, in particular linear or branched C₁₋₄ alkyl. This alkyl may have 1 to 6, particularly 1 to 3, substituents, such as aryl and alkoxy described below.

Examples of aryl represented by R¹ in formula (1) include phenyl, pentalenyl, naphthyl, anthracenyl, tetracenyl, pentacenyl, phenanthrenyl, fluoranthenyl, and coronenyl. This aryl may have 1 to 6, particularly 1 to 3, substituents, such as the alkyl, the aryl, and alkoxy described below.

Alkoxy represented by R¹ in formula (1) includes linear or branched C₁₋₆ alkoxy such as methoxy, ethoxy, n-propoxy, isopropoxy, n-butyloxy, isobutyloxy, sec-butyloxy, tert-butyloxy, n-pentyloxy, and n-hexyloxy, in particular linear or branched C₁₋₄ alkoxy. This alkoxy may have 1 to 6, particularly 1 to 3, substituents, such as the alkyl, aryl, and alkoxy.

In particular, R¹ is preferably a hydrogen atom from the standpoint of simplicity of synthesis, yield, and stability of the carbon nanobelt of the present invention, for example. R¹ is preferably alkoxy from the standpoint of solubility, stability, and film formability of the carbon nanobelt of the present invention, for example.

The carbon nanobelt according to the present invention may be, for example, a compound represented by formula (1A):

wherein Ar and n are as defined above, and R¹ groups are all hydrogen. This carbon nanobelt represented by formula (1A) wherein R¹ groups are all hydrogen is a compound having a ring structure composed of repeating units represented by formula (6A):

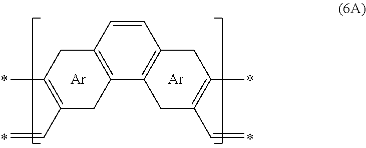

wherein Ar is as defined above, and the asterisk “*” represents a binding site.

Examples of the aromatic hydrocarbon ring represented by Ar in formula (1) include a benzene ring, a naphthalene ring, an anthracene ring, a tetracene ring, a pentacene ring, a phenanthrene ring, a benzanthracene ring, a pyrene ring, a perylene ring, a triphenylene ring, a phenalene ring, a fluoranthene ring, and a coronene ring. Of these, a benzene ring and a naphthalene ring are preferable, and a benzene ring is more preferable, from the standpoint of simplicity of synthesis, yield, stability of the carbon nanobelt of the present invention, for example. The aromatic hydrocarbon ring represented by Ar may also be optionally substituted. Examples of the substituent that the aromatic hydrocarbon ring represented by Ar may have include the alkyl described above, the aryl described above, and the alkoxy described above. When the aromatic hydrocarbon ring is substituted, the number of substituents is preferably 1 to 6, and more preferably 1 to 3. The aromatic hydrocarbon ring represented by Ar in formula (1) may be one type or a combination of two or more types.

In formula (1), n represents an integer of 0 or more, and from the standpoint of simplicity of synthesis and yield, for example, n preferably represents an integer of 0 to 10. In particular, from the standpoint of stability of the carbon nanobelt of the present invention, n represents an integer of 1 to 6, and more preferably an integer of 1 to 4.

In view of the descriptions above, the carbon nanobelt according to the present invention preferably has 2 to 12 repeating units represented by formula (6), more preferably 3 to 8 repeating units represented by formula (6), and most preferably 3 to 6 repeating units represented by formula (6).

Specifically, particularly preferable carbon nanobelts include those described below.

The carbon nanobelt according to the present invention can also be obtained as a solvate of, for example, an aliphatic halogenated hydrocarbon, such as dichloromethane, chloroform, carbon tetrachloride, and dichloroethane.

The carbon nanobelt according to the present invention is considered to be useful as a stable template or scaffold for selectively synthesizing a carbon nanotube having a uniform diameter.

### 2. Method for Producing Carbon Nanobelt

The carbon nanobelt according to the present invention is produced, for example, by a production method comprising reacting, in the presence a nickel catalyst, a cyclic compound represented by formula (2):

wherein R¹ and n are as defined above; each Ar′ is identical or different and represents an aromatic hydrocarbon group having two halogen atoms as substituents; and R¹ represents hydrogen, alkyl, aryl, or alkoxy.

In formula (2), each Ar′ represents an aromatic hydrocarbon group having two halogen atoms as substituents. Specifically, Ar′ is structured as the aromatic hydrocarbon ring described above having two halogen atoms bound to it. Ar′ is optionally substituted with other substituents of the alkyl described above, the aryl described above, or the alkoxy described above; and the number of substituents is preferably 1 to 6.

Specifically, such Ar′ is preferably a group represented by formula (5A) or (5B):

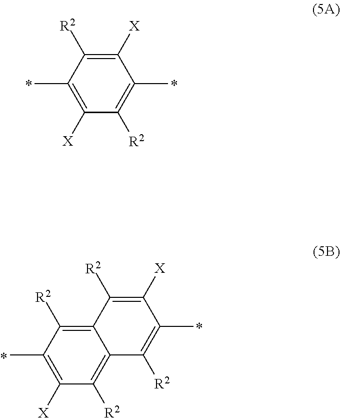

wherein each R² is identical or different and represents hydrogen, alkyl, aryl, or alkoxy; X is identical or different and represents a halogen atom; and the asterisk “*” represents a binding site.

In formulas (5A) and (5B), examples of the halogen atom represented by X include fluorine, chlorine, bromine, and iodine. From the standpoint of simplicity of synthesis and yield, for example, the halogen atom is preferably bromine or iodine, and more preferably bromine. Additionally, each X is preferably identical.

In formulas (5A) and (5B), alkyl, aryl, and alkoxy represented by R² are as defined above. The type and the number of substituents are also as defined above.

Examples of such Ar′ include those as described below.

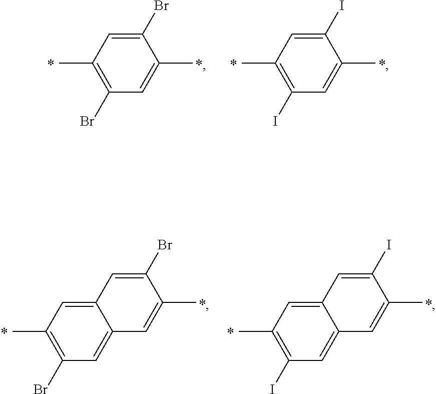

### (2-1) Cyclic Compound (2)

The cyclic compound represented by formula (2) is preferably a compound wherein R¹ is hydrogen, and represented by formula (2A):

wherein Ar′ and n are as defined above. This compound is more preferably a compound wherein n is 1 to 4. In particular, this compound is particularly preferably a compound wherein Ar′ represents a benzene ring or a naphthalene ring.

Such a cyclic compound represented by formula (2) is a novel compound that has yet to been disclosed in literature, and examples include those described below.

This cyclic compound represented by formula (2) can be obtained as a solvate of, for example, an aromatic hydrocarbon, such as benzene, toluene, xylene, and chlorobenzene. A method for producing these compounds represented by formula (2) is described later.

### (2-2) Nickel Catalyst

The nickel catalyst is not particularly limited, and is preferably a salt of non-valent Ni or a salt of divalent Ni.

The salt of non-valent Ni is not particularly limited, and includes bis(1,5-cyclooctadiene)nickel(0) (Ni(C₈H₁₂)₂), bis(triphenylphosphine)nickel dicarbonyl, and nickel carbonyl.

Examples of the salt of divalent Ni include nickel(II) acetate, nickel(II) trifluoroacetate, nickel(II) nitrate, nickel(II) chloride, nickel(II) bromide, nickel(II) acetylacetonato, nickel(II) perchlorate, nickel(II) citrate, nickel(II) oxalate, nickel(II) cyclohexanebutyrate, nickel(II) benzoate, nickel(II) stearate, nickel(II) stearate, nickel(II) sulfamate, nickel(II) carbonate, nickel(II) thiocyanate, nickel(II) trifluoromethanesulfonate, nickel(II) bis(1,5-cyclooctadiene), nickel(II) bis(4-diethylaminodithiobenzil), nickel(II) cyanide, nickel(II) fluoride, nickel(II) boride, nickel(II) borate, nickel(II) hypophosphite, nickel(II) ammonium sulfate, nickel(II) hydroxide, cyclopentadienyl nickel(II), hydrates thereof, and mixtures thereof.

The salt of non-valent Ni and the salt of divalent Ni for use may be a nickel catalyst obtained by binding a known ligand by a coordinate bond in accordance with an ordinary method.

Typically, the amount of the nickel catalyst for use is preferably 3 to 50 mols, and more preferably 5 to 20 mols, per mol of the compound represented by formula (2) (a starting material), from the standpoint of, for example, simplicity of synthesis and yield.

### (2-3) Ligand Compound

For this reaction, a ligand compound that can bind to nickel by coordinate bond may be used together with the nickel catalyst. This ligand compound includes those whose coordinating atom is nitrogen, phosphorus, oxygen, sulfur, etc. These ligand compounds include unidentate ligands having only one coordinating atom and polydentate ligands having two or more coordinating atoms.

Examples of unidentate ligands include triphenylphosphine, trimethoxyphosphine, triethylphosphine, triisopropylphosphine, tri(tert-butyl)phosphine, tri(n-butyl) phosphine, triisopropoxyphosphine, tricyclopentylphosphine, tricyclohexylphosphine, di(tert-butyl)methylphosphine, methyldiphenylphosphine, dimethylphenylphosphine, triethylamine, and pyridine.

Examples of polydentate ligands include 2,2′-bipyridyl, 4,4′-(tert-butyl)bipyridyl, 4,4′-bis(trifluoromethyl)-2,2′-bipyridyl, 5,5′-bis(trifluoromethyl)-2,2′-bipyridyl, 6,6′-bis(trifluoromethyl)-2,2′-bipyridyl, 4,4′-bis(methoxycarbonyl)-2,2′-bipyridyl, 4,4′-dimethyl-2,2′-bipyridyl, 5,5′-dimethyl-2,2′-bipyridyl, 4,4′-dimethoxy-2,2′-bipyridyl, 4,4′-dicyano-2,2′-bipyridyl, phenanthroline, 2,2′-bipyrimidyl, 1,4-diazabicyclo[2.2.2]octane, 2-(dimethylamino)ethanol, tetramethylethylenediamine, N,N-dimethylethylene diamine, N,N′-dimethylethylene diamine, 2-amino methyl pyridine, 1,1′-bis(diphenylphosphino)ferrocene, diphenylphosphinomethane, 1,2-bis(diphenylphosphino)ethane, 1,3-bis(diphenylphosphino)propane, 1,5-bis(diphenylphosphino)pentane, 1,2-bis(dicyclohexylphosphino)ethane, 1,3-(dicyclohexylphosphino)propane, 1,2-bis(di-tert-butylphosphino)ethane, 1,3-bis(di-tert-butylphosphino)propane, 1,2-bis(diphenylphosphino)benzene, 1,5-cyclooctadiene, 2,2′-bis(diphenylphosphino)-1,1′-binaphthyl (BINAP), 2,2′-dimethyl-6,6′-bis(diphenylphosphino)biphenyl (BIPHEMP), 1,2-bis(diphenylphosphino)propane (PROPHOS), 2,3-O-isopropylidene-2,3-dihydroxy-1,4-bis(diphenylphosphino)butane (DIOP), 3,4-bis(diphenylphosphino)-1-benzylpyrrolidine (DEGUPHOS), 1,2-bis[(2-methoxyphenyl)phenylphosphino]ethane (DIPAMP), substituted-1,2-bis phospholano benzene (DuPHOS), 5,6-bis(diphenylphosphino)-2-norbornene (NORPHOS), N,N′-bis(diphenylphosphino)-N,N′-bis(1-phenylethyl)ethylene diamine (PNNP), 2,4-bis(diphenylphosphino)pentane (SKEWPHOS), 1-[1′,2-bis(diphenylphosphino)ferrocenyl]ethylene diamine (BPPFA), 2,2′-bis(dicyclohexylphosphino)-5,5′,6,6′,7,7′,8,8′-octahydro-1,1′-binaphthyl, ((4,4′-bi-1,3-benzodioxole)-5,5′-diyl)bis(diphenyl phosphine)(SEGPHOS), 2,3-bis(diphenylphosphino)butane (CHIRAPHOS), and 1-[2-(2-substituted phosphino)ferrocenyl]ethyl-2 substituted phosphine (JOSIPHOS), and mixtures thereof. BINAP includes derivatives of BINAP(2,2′-bis(diphenylphosphino)-1,1′-binaphthyl), and BIPHEMP includes derivatives of BIPHEMP(2,2′-dimethyl-6,6′-bis(diphenylphosphino)biphenyl). Of the ligands, from the standpoint of simplicity of synthesis and yield, for example, a polydentate ligand is preferable. 2,2′-bipyridyl, 4,4′-(tert-butyl)bipyridyl, 4,4′-bis(trifluoromethyl)-2,2′-bipyridyl, 5,5′-bis(trifluoromethyl)-2,2′-bipyridyl, 6,6′-bis(trifluoromethyl)-2,2′-bipyridyl, 4,4′-bis(methoxycarbonyl)-2,2′-bipyridyl, 4,4′-dimethyl-2,2′-bipyridyl, 5,5′-dimethyl-2,2′-bipyridyl, 4,4′-dimethoxy-2,2′-bipyridyl, 4,4′-dicyano-2,2′-bipyridyl, and the like are more preferable. 2,2′-bipyridyl, 4,4′-bis(trifluoromethyl)-2,2′-bipyridyl, 5,5′-bis(trifluoromethyl)-2,2′-bipyridyl, 4,4′-bis(methoxycarbonyl)-2,2′-bipyridyl, 4,4′-dimethyl-2,2′-bipyridyl, 5,5′-dimethyl-2,2′-bipyridyl, and 4,4′-dimethoxy-2,2′-bipyridyl are still more preferable.

When a ligand compound is used, the amount of the ligand compound for use is preferably 0.2 to 5 mols, and more preferably 0.5 to 2 mols, per mol of the nickel catalyst.

### (2-4) Reaction

The reaction can be performed in the presence of a typical solvent. Examples of the reaction solvent for use include aliphatic hydrocarbons, such as n-hexane, cyclohexane, and n-heptane; aliphatic halogenated hydrocarbons, such as dichloromethane, chloroform, carbon tetrachloride, and dichloroethane; aromatic hydrocarbons, such as benzene, toluene, xylene, and chlorobenzene; ethers, such as diethyl ether, diisopropyl ether, di-n-butyl ether, dimethoxyethane (DME), cyclopentyl methyl ether (CPME), tert-butyl methyl ether, tetrahydrofuran (THF), and dioxane; esters, such as ethyl acetate, and ethyl propionate; amides, such as dimethylformamide (DMF), N,N-dimethylacetamide (DMA), and N-methylpyrrolidone (NMP); nitriles, such as acetonitrile, and propionitrile; and dimethyl sulfoxide (DMSO). These solvents may be used singly or in a combination of two or more. Of these, amides are preferable, and dimethylformamide (DMF) is more preferable.

Typically, the reaction is preferably performed in an inert gas (e.g., nitrogen or argon) atmosphere. To reduce or prevent an unexpected reaction, such as isomerization, the reaction is preferably performed in a dark room.

Regarding the reaction temperature, the reaction may be performed with heating, at room temperature, or with cooling. Typically, the reaction temperature is preferably 50 to 100° C. The reaction time is not particularly limited, and is, for example, 1 minute to 24 hours.

After completion of the reaction, a typical isolation technique, such as filtration, concentration, and/or extraction, is optionally performed; and a typical purification technique, such as column chromatography and/or recrystallization, is optionally performed, thereby isolating and purifying the carbon nanobelt according to the present invention.

### 3. Method for Producing Cyclic Compound (2)

The cyclic compound (2) according to the present invention is produced, for example, by a production method comprising reacting, in the presence of a base, a compound represented by formula (3A) or a salt thereof:

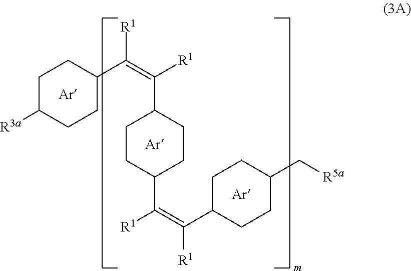

wherein Ar′ and R¹ are as defined above, R^{5a} represents triaryl phosphine or triaryl phosphite, m represents an integer of 0 or more, and R^{3a} represents acyl or a group represented by formula (4A), with the proviso that when m is 0, R^{3a} is the group represented by formula (4A):

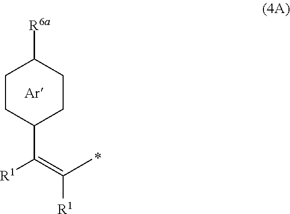

wherein Ar′ and R¹ are as defined above, and R^{6a} represents acyl. Specifically, the cyclic compound (2) is obtained by a Wittig reaction.

### (3-1) Compound (3A)

Examples of triaryl phosphine represented by R^{6a} in formula (3A) include triphenylphosphine, trimesitylphosphine, tris(2-methylphenyl)phosphine, and tris(2,4,6-tricyclohexylphenyl)phosphine. Examples of triaryl phosphite represented by R^{5a} include triphenyl phosphite, tris(2-methylphenyl)phosphite, tris(2,6-dimethylphenyl)phosphite, and tris(2,4-di-tert-butylphenyl)phosphite.

In formula (3A), m is an integer of 0 or more, and from the standpoint of simplicity of synthesis and yield, for example, m is preferably an integer of 0 to 5. In particular, from the standpoint of the stability of the carbon nanobelt of the present invention, m is preferably an integer of 1 to 3, and more preferably 1 or 2.

In formulas (3A) and (4A), examples of acyl represented by R^{3a} or R^{6a} include linear or branched C₁₋₆ alkanoyl, such as formyl, acetyl, and propionyl.

When a salt of a compound represented by formula (3A) is used, the compound represented by formula (3A) is likely to become a cation. Examples of a paired anion for the cation include halogen ions (a fluorine ion, a chlorine ion, a bromine ion, and an iodine ion), and a hexafluorophosphate anion (PF₆₋).

Examples of a compound represented by formula (3A) or a salt thereof that satisfies such conditions include those described below:

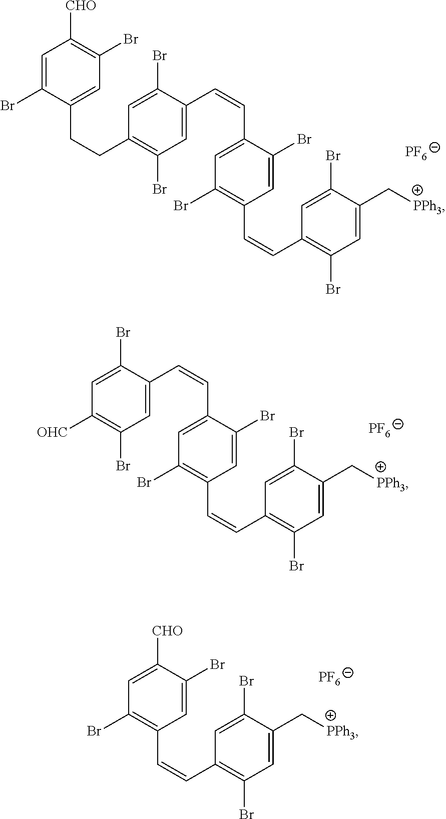

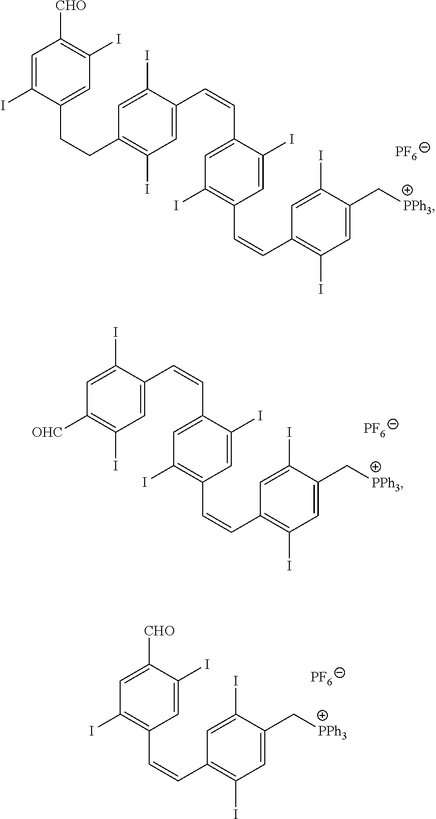

wherein Ph represents phenyl; the same applies below.

The cyclic compound represented by formula (3A) can be obtained as a solvate of, for example, an ester, such as ethyl acetate, and ethyl propionate. A method for producing these compounds represented by formula (3A) is described later.

### (3-2) Base

Examples of bases include alkali metal hydroxides, such as lithium hydroxide, sodium hydroxide, and potassium hydroxide; and alkali metal alkoxides, such as potassium tert-butoxide, and sodium methoxide.

Typically, the amount of a base for use is preferably 0.2 to 5 mols, and more preferably 0.5 to 2 mols, per mol of the compound represented by formula (3A).

### (3-3) Reaction

The reaction can be performed in the presence of a typical solvent. Examples of the reaction solvent for use include aliphatic hydrocarbons, such as n-hexane, cyclohexane, and n-heptane; aliphatic halogenated hydrocarbons, such as dichloromethane, chloroform, carbon tetrachloride, and dichloroethane; aromatic hydrocarbons, such as benzene, toluene, xylene, and chlorobenzene; ethers, such as diethyl ether, diisopropyl ether, di-n-butyl ether, dimethoxyethane (DME), cyclopentyl methyl ether (CPME), tert-butyl methyl ether, tetrahydrofuran (THF), and dioxane; esters, such as ethyl acetate, and ethyl propionate; amides, such as dimethylformamide (DMF), N,N-dimethylacetamide (DMA), and N-methylpyrrolidone (NMP); nitriles, such as acetonitrile, and propionitrile; and dimethyl sulfoxide (DMSO). These solvents can be used singly or in a combination of two or more. Of these, aliphatic halogenated hydrocarbons are preferable, and dichloromethane is more preferable.

Typically, the reaction is preferably performed in an inert gas (e.g., nitrogen and argon) atmosphere. To reduce or prevent an unexpected reaction, such as isomerization, the reaction is preferably performed in a dark room.

Regarding the reaction temperature, the reaction may be performed with heating, at room temperature, or with cooling. Typically, the reaction temperature is preferably 0 to 50° C. The reaction time is not particularly limited, and is, for example, 1 minute to 24 hours.

After completion of the reaction, a typical isolation technique, such as filtration, concentration, and/or extraction, is optionally performed; and a typical purification technique, such as column chromatography and/or recrystallization, is optionally performed, thereby isolating and purifying the compound represented by formula (2). Alternatively, after completion of the reaction, a synthesis step for synthesizing the carbon nanobelt according to the present invention may be performed without such isolation and purification.

### 4. Method for Producing Compound (3A)

In synthesis of compound (3A) according to the present invention, compound (3B) is first synthesized through, for example, reaction scheme 1:

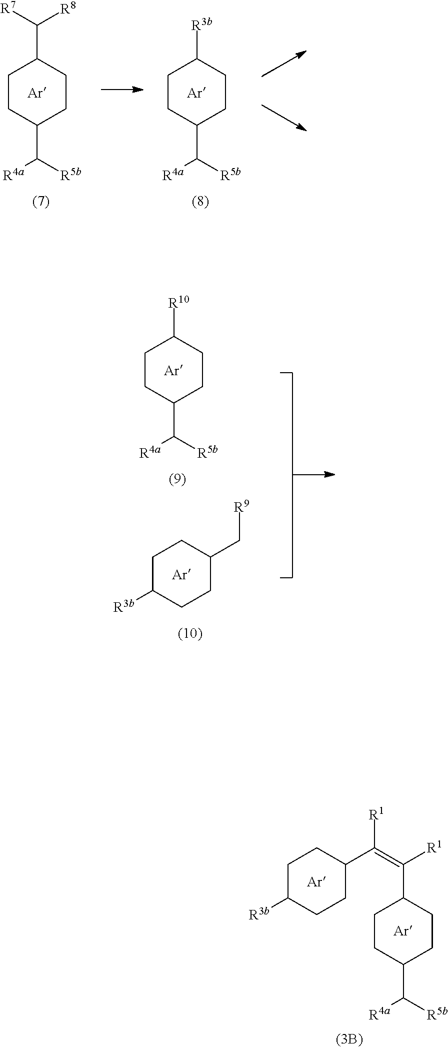

wherein R¹ are as defined above; R^{3b} represents dialkoxymethyl; R^{4a}, R^{5b}, R⁷, R⁸, and R⁹ are identical or different and represent a halogen atom; and R¹⁰ represents acyl.

In formulas (8), (10), and (3B), dialkoxymethyl represented by R^{3b} includes dimethoxy methyl and diethoxy methyl.

In formulas (7), (8), (9), (10), and (3B), examples of the halogen atom represented by R^{4a}, R^{5b}, R⁷, R⁸, or R⁹ include fluorine, chlorine, bromine, and iodine. From the standpoint of simplicity of synthesis and yield, for example, the halogen atom is preferably bromine or iodine, and more preferably bromine.

In formula (9), examples of acyl represented by R′° include linear or branched C₁₋₆ alkanoyl, such as formyl, acetyl, and propionyl.

Specifically, compound (7) obtained in accordance with a known method is reacted with a base (e.g., alkali metal alkoxides, such as potassium tert-butoxide and sodium methoxide) to obtain compound (8). While compound (8) is reacted with an acid (e.g., hydrochloric acid) to obtain compound (9), compound (8) is reacted with dialkyl phosphite (e.g., dimethyl phosphite) and a base (e.g., N,N-diisopropylethylamine) to obtain compound (10). Subsequently, compound (9) is reacted with compound (10) in the presence of triaryl phosphine (e.g., triphenylphosphine) and a base (e.g., alkali metal alkoxides, such as potassium tert-butoxide and sodium methoxide) to obtain compound (3B). The amount of reagents and conditions for each step can follow ordinary methods.

Subsequently, compound (3A) is synthesized through reaction scheme 2:

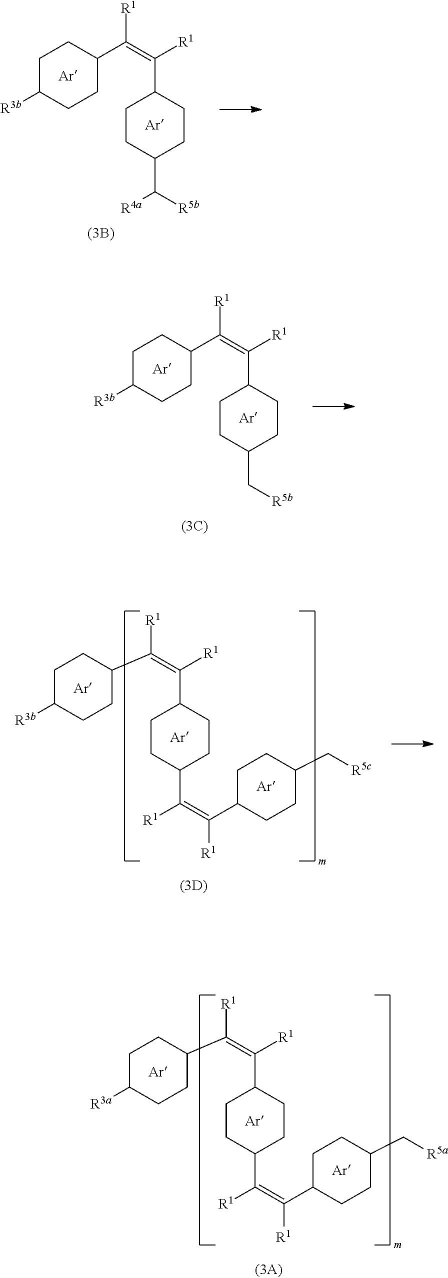

wherein Ar′, R¹, R^{3a}, R^{3b}, R^{4a}, R^{5a}, R^{5b}, and m are as defined above; and each R^{5c} is identical or different and represents a halogen atom.

In formula (3D), examples of the halogen atom represented by R^{5c} include fluorine, chlorine, bromine, and iodine; and from the standpoint of simplicity of synthesis and yield, for example, the halogen atom is preferably bromine, iodine, etc., and more preferably bromine.

Specifically, compound (3B) obtained by reaction scheme 1 is reacted with dialkyl phosphite (e.g., dimethyl phosphite) and a base (e.g., ethyldiisopropylamine) to obtain compound (3C). Compound (3C) is reacted with a suitable compound in the presence of triaryl phosphine (e.g., triphenylphosphine) and a base (e.g., alkali metal alkoxides, such as potassium tert-butoxide and sodium methoxide) to obtain compound (3D). For example, to obtain compound (3D) wherein m is 1, compound (3C) is reacted with compound (9). To obtain compound (3A) wherein m is 2 or more, adjustment can be suitably made with reference to this method. Finally, the obtained compound (3D) is reacted with dialkyl phosphite (e.g., dimethyl phosphite) and a base (e.g., ethyldiisopropylamine) to obtain compound (3A). The amount of reagents and conditions for each step can follow ordinary methods.

In this reaction scheme 2, compounds (3A), (3B), (3C), and (3D) are all novel compounds that have yet to be disclosed in literature, and are collectively represented by formula (3):

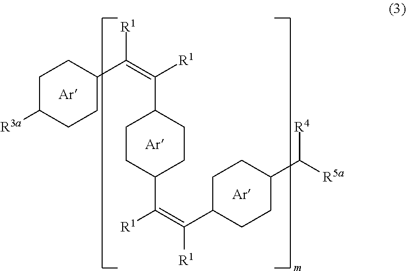

wherein Ar′, R¹, and m are as defined above; R⁴ represents a hydrogen atom or a halogen atom; R⁵ represents a halogen atom, triaryl phosphine, or triaryl phosphite; and R³ represents acyl, dialkoxymethyl, or a group represented by formula (4), with the proviso that when m is 0, R³ is the group represented by formula (4):

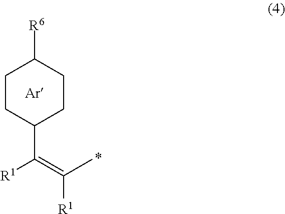

wherein Ar′ and R¹ are as defined above, and R⁶ represents acyl or dialkoxymethyl.

### EXAMPLES

The following describes the present invention in detail with reference to Examples. However, the present invention is not limited to these Examples.

Unless otherwise indicated, all of the reactions were performed in a dry glass container in a nitrogen atmosphere using a dry solvent in accordance with a standard vacuum line technique. Unless otherwise restricted, all of the materials were obtained from commercial suppliers and used without being purified. Triphenylphosphine (PPh₃) was recrystallized from n-hexane, and N,N-diisopropylethylamine (DIPEA) was filtered through short-pad basic aluminum oxide. A solution of potassium tert-butoxide in tetrahydrofuran (THF) was titrated with 1N HCl before use. Tetrahydrofuran, dichloromethane, and toluene for use in a reaction were purified with an organic solvent purifier (Glass Contour). Unless otherwise indicated, all of the treatment and purification procedures were performed in air with a reagent grade solvent. For isolation of [12]ICP (compound 11), n-hexane, dichloromethane, and toluene were redistilled before use; and 1,3-dimethyl-3,4,5,6-tetrahydro-2-pyrimidinone (DMPU), N,N-dimethylacetamide (DMA), and CHCl₃ were of reagent grade and HPLC grade.

Thin-layer chromatography (TLC) was performed using a plate (0.25 mm) pre-coated with E. Merck silica gel 60 F254. Chromatograms were analyzed with a UV lamp (254 nm and 365 nm) or by immersion in a solution of 2,4-di-nitro phenyl hydrazine (12 g) and H₂SO₄ (60 mL) in water/ethanol (2:5, 280 mL) and heating. Flash column chromatography was performed using E. Merck silica gel 60 (230-400 mesh). Melting point was measured with an MPA100 Optimelt melting point measurement apparatus. High-resolution mass spectrometry (HRMS) was measured with Bruker Daltonics Ultraflex III TOF/TOF (MALDI-TOF-MS) or JEOL JMS-S3000 SpiralTOF (MALDI-TOF MS), using a polypropylene glycol mixture (PPG) as an internal standard and trans-2-[3-(4-tert-butylphenyl)-2-methyl-2-propenylidene]malononitrile (DCTB) as a matrix containing NaI as a cationizing agent. Analysis of benzyl bromide (compounds 3, 6, and 8) was not performed; instead, the precise mass of a corresponding triphenyl phosphonium adduct was measured. Nuclear magnetic resonance (NMR) spectra were recorded with a JEOL spectrometer (JNM-ECA-600: ¹H 600 MHz, ¹³C 150 MHz; JEOL ECA 50011: ¹H 500 MHz, ¹³C 125 MHz; JEOL JNM-A-400: ¹H 400 MHz, ¹³C 100 MHz). The chemical shift of ¹H NMR was shown in parts per million (ppm) relative to CHCl₃ (δ7.26 ppm) or CHDCl₂ (δ5.32 ppm). ¹³C NMR spectra were measured with a proton decoupling pulse sequence. The chemical shift of ¹³C NMR was shown in parts per million (ppm) relative to CDCl₃ (δ77.0 ppm) or CD₂Cl₂ (δ53.8 ppm). The following abbreviations were used for describing multiplicity: s (singlet), d (doublet), t (triplet), sept (septuplet), and m (multiplet). When different coupling constants were equal by chance, such a case was shown as “app” (apparent). A coupling constant J was reported in the unit of Hz, with accuracy to the last digit. When possible, multiplicity was interpreted to the second order according to Pople's nomenclature. When the resolution was not clearly interpreted, some signals were approximated to the first order.

The following describes Examples concerning a carbon nanobelt (an isocyclophenacene compound). An isocyclophenacene compound wherein n is an integer of 0 or more, or a derivative thereof, can be synthesized in the same manner. In the following Examples, “[n]ICP” refers to an isocyclophenacene compound wherein the number of rings is n.

### Synthesis of [12]Isocyclophenacene ([12]ICP)

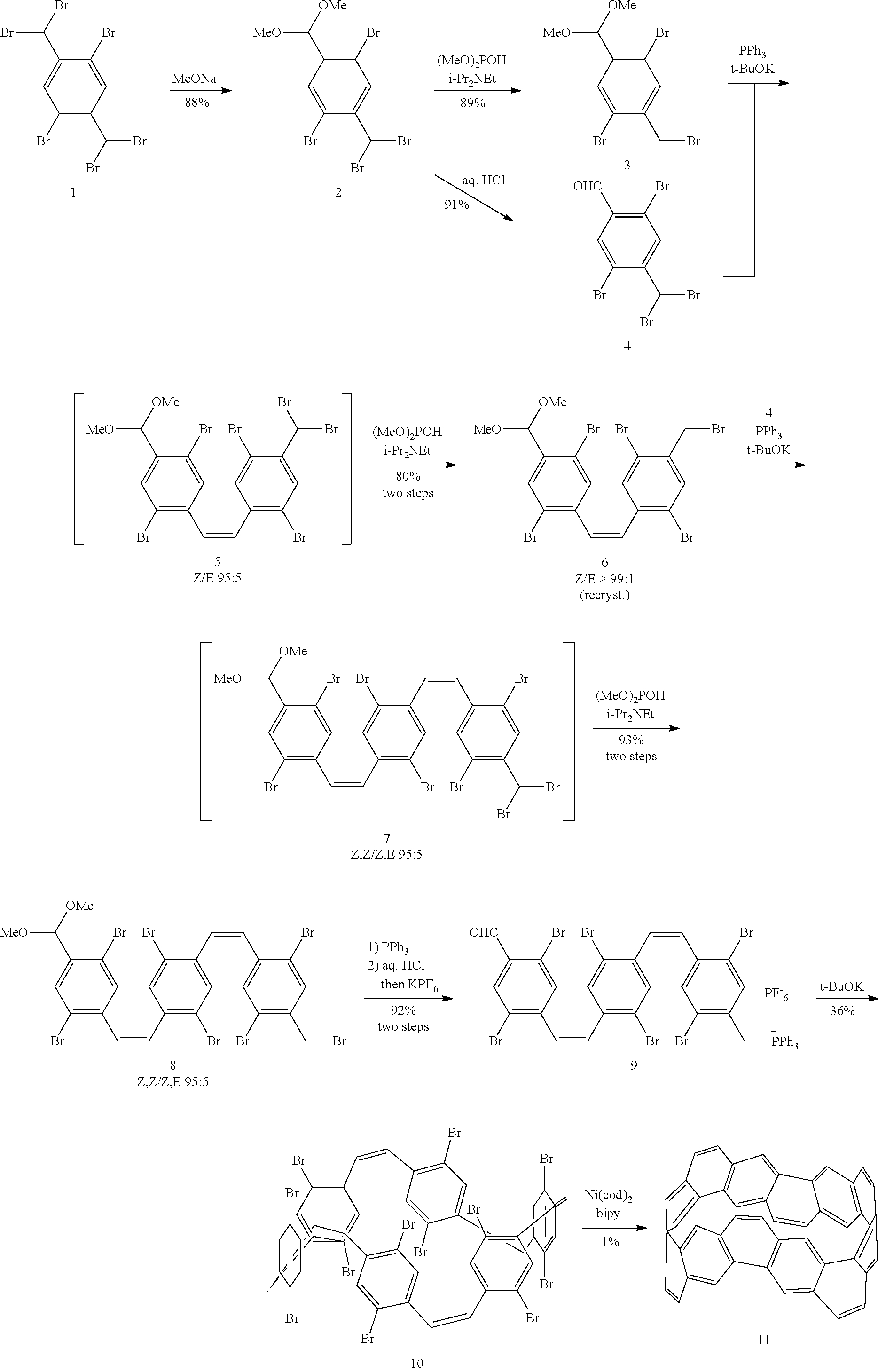

wherein Me represents methyl, i-Pr represents isopropyl, Et represents ethyl, t-Bu represents tert-butyl, cod represents 1,5-cyclooctadiene, and bipy represents 2,2′-bipyridyl.

### Notes

When exposed to light, a solution of a Z-stilbene derivative, including cyclic compound 10, changed in color to yellow and was inclined to exhibit an unexpected photoreaction process, such as isomerization. Although the degradation rate of cyclic compound 10 appeared relatively slow, glass products were shielded with aluminum foil as much as possible to perform an experiment. The obtained [12]ICP (compound 11) was especially sensitive to light, and care was required during an operation in a solution. However, after crystallization, [12]ICP was stable for a long time even when exposed to ambient atmosphere and light.

### Synthesis Example 1: 2,5-dibromo-4-dibromomethyl benzaldehyde dimethyl acetal (compound 2)

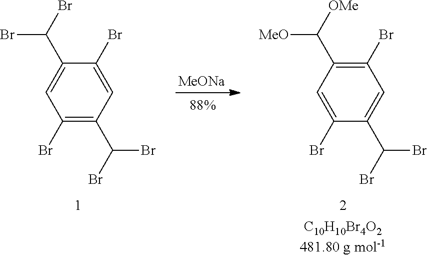 [SMILES: "MeONa" ; Brc1cc(C(Br)Br)c(Br)cc1C(Br)Br ; COC(OC)c1cc(Br)c(C(Br)Br)cc1Br]

Compound 1 was synthesized in accordance with a published report (Chem. Int. Ed., 2014, 53, 6786).

1,4-dibromo-2,5-bis(dibromomethyl)benzene (compound 1; 5.80 g, 10.0 mmol, 1 equivalent) was added in a nitrogen atmosphere to a newly prepared solution of sodium methoxide in methanol (2.6 M, 20 mL; a degreased sodium metal not containing oxides (1.21 g, 52.6 mmol, 5.26 equivalents) was added to 20 mL of dry methanol), and then dry toluene (50 mL) was added thereto. Subsequently, a condenser was attached to a reactor, and the heterogeneous mixture was refluxed (oil bath temperature: about 100° C.). After 45 minutes, the starting materials were confirmed to have completely disappeared by TLC analysis (hexane/ethyl acetate, 9:1). The reaction mixture was cooled to room temperature, and then neutralized with a saturated NH₄Cl aqueous solution and a minimum amount of water to dissolve inorganic salts. The obtained biphasic, transparent solution was extracted with n-hexane three times, and the combined organic layer was washed with brine, followed by drying over Na₂SO₄. The result was then filtered and concentrated under vacuum, thereby obtaining 4.8 g of a brown oil. The crude product was purified by flash column chromatography (hexane/ethyl acetate, 100:0 to 97:3, with a mild gradient), thereby obtaining compound 2 as a white solid (4.25 g, 88%).

The same process was performed using compound 1 (81.1 g, 140 mmol, 1 equivalent), a solution of sodium methoxide in methanol (about 3M, 230 mL; a degreased sodium metal not containing oxides (16.4 g, 713 mmol, 5.09 equivalents) was added to 230 mL of dry methanol), and toluene (1.15 L). The mixture was refluxed for 2.5 hours, thereby obtaining 55 g (81%) of compound 2 (large scale) in the same manner.

Acetal 2 can be recrystallized from hot n-hexane as white fine needles; m.p.: 70-71° C. ¹H NMR (400 MHz, CDCl₃) δ 8.18 (s, 1H), 7.74 (s, 1H), 6.96 (s, 1H), 5.48 (s, 1H), 3.39 (s, 6H). ¹³C NMR (100 MHz, CDCl₃) δ 141.9, 140.0, 135.2, 132.3, 122.4, 118.5, 101.8, 54.1. (2C), 37.9. Anal. Calcd for C₁₀H₁₀Br₄O₂: C, 24.93; H, 2.09; Br, 66.34; O, 6.64. Found: C, 24.89; H, 2.08.

### Synthesis Example 2: 2,5-dibromo-4-bromomethyl benzaldehyde dimethyl acetal (compound 3)

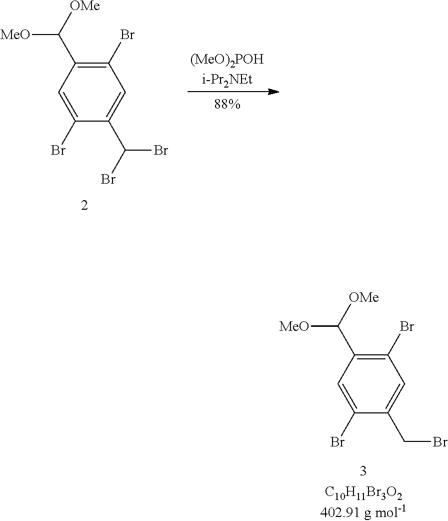 [SMILES: "(MeO)2POHi-Pr2NEt" ; COC(OC)c1cc(Br)c(C(Br)Br)cc1Br ; COC(OC)c1cc(Br)c(CBr)cc1Br]

2,5-dibromo-4-dibromomethyl benzaldehyde dimethyl acetal (compound 2, 1.70 g, 3.54 mmol) in THF (15 mL) was cooled in an ice bath. Subsequently, N,N-diisopropylethylamine (DIPEA; 0.90 mL, 5.2 mmol, 1.5 equivalents) and dimethyl phosphite (0.45 mL, 4.9 mmol, 1.4 equivalents) were added thereto. After the obtained transparent mixture was stirred at the same temperature for 90 minutes, fine precipitates were formed. Compound 2 was then confirmed to have completely disappeared by TLC (hexane/ethyl acetate, 9:1) analysis. The obtained mixture was diluted with n-hexane, and dilute hydrochloric acid (about 0.2M, 50 mL) was added thereto to protonate the excessive base. The layers were separated, and the aqueous phase was extracted with n-hexane two times or more. Subsequently, the combined organic layer was continuously washed with a saturated NaHCO₃ aqueous solution and brine, dried over Na₂SO₄, and filtrated, followed by concentration under vacuum. Flash column chromatography (n-hexane/ethyl acetate 100:0 to 97:3) was performed to obtain the target compound 3 as a colorless oil, which was then allowed to stand under high vacuum, thereby obtaining a white solid (1.25 g, 88%).

The same process was performed using compound 2 (44.0 g, 91.3 mmol, 1 equivalent) in THF (150 mL), dimethyl phosphite (11.6 mL, 126 mmol, 1.39 equivalents), and DIPEA (23.2 mL, 133 mmol, 1.46 equivalents). The mixture as stirred at 0° C. for 1.5 hours, thereby obtaining 32.7 g (89%) of compound 3 (large scale) in the same manner.

Amorphous white solid, m.p.: 65-66° C. ¹H NMR (400 MHz, CDCl₃) δ 7.81 (s, 1H), 7.65 (s, 1H), 5.48 (s, 1H), 4.52 (s, 2H), 3.38 (s, 6H). ¹³C NMR (100 MHz, CDCl₃) δ 138.84, 138.81, 135.0, 133.0, 123.2, 121.6, 101.8, 53.9 (2C), 31.7. HRMS of the triphenyiphosphine adduct; m/z [C₂₈H₂₆(⁷⁹Br)(⁸¹Br)O₂P]⁺ calcd.: 585,0012, found: 585.002.

### Synthesis Example 3: 2,5-dibromo-4-dibromomethyl benzaldehyde (Compound 4)

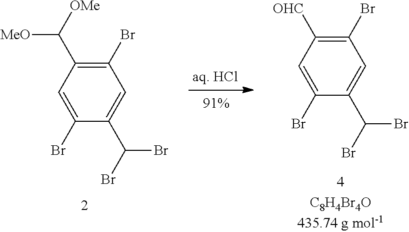 [SMILES: COC(OC)c1cc(Br)c(C(Br)Br)cc1Br ; "aq. HCl" ; O=Cc1cc(Br)c(C(Br)Br)cc1Br]

Hydrochloric acid (3M, 5 mL) was added to 2,5-dibromo-4-dibromomethyl benzaldehyde dimethyl acetal (compound 2; 1.04 g, 2.16 mmol, 1 equivalent) in THF (10 mL). The obtained biphasic mixture was stirred under reflux (oil bath: 75° C.) for 1 hour, and complete conversion was then confirmed by TLC (n-hexane/ethyl acetate, 9:1). The reaction mixture was cooled to room temperature and diluted with n-hexane, followed by neutralization with a saturated NaHCO₃ aqueous solution. The layers were separated, and the aqueous phase was extracted with n-hexane twice. Subsequently, the combined organic layer was washed with brine, dried over Na₂SO₄, and filtered, followed by concentration under vacuum. The solid residue was dried under high vacuum to remove the trace of THF (to remove poorly soluble 2,5-dibromoterephthalaldehyde formed as a by-product by thermal filtration), and recrystallization from n-hexane was performed. After performing recrystallization (3 times), colorless to pale yellow-green compound 4 was obtained as fine platelets. However, the integration of a ¹H NMR spectrum found that 2 to 3% of 2,5-dibromoterephthalaldehyde was present (0.873 g, 91% corrected).

The same process was performed using compound 2 (64.0 g, 133 mmol, 1 equivalent) in THF (300 mL), and hydrochloric acid (6M, 150 mL). The mixture was stirred under reflux for 1 hour, and recrystallization from n-hexane (300 mL) was performed, thereby obtaining 48.5 g (84%) of compound 4 in the same manner (large scale).

Colorless to pale yellow-green fine platelets, m.p.: 93.5-94.5° C. (n-hexane). ¹H NMR (400 MHz, CDCl₃) δ 10.27 (s, 1H), 8.28 (s, 1H), 8.03 (s, 1H), 6.95 (s, 1H). ¹³C NMR (100 MHz, CDCl₃) δ 189.4, 146.6, 136.2, 134.7, 133.8, 125.8, 119.4, 37.1. Anal. Calcd for C₈H₄Br₂O: C, 22.05; H, 0.93; Br, 73.35; 0, 3.67. Found: C, 22.30; H, 1.00.

### Example 1: (Z)-2,5-dibromo-4-(2,5-dibromo-4-(bromomethyl)styryl)benzaldehyde dimethyl acetal (Compound 6)

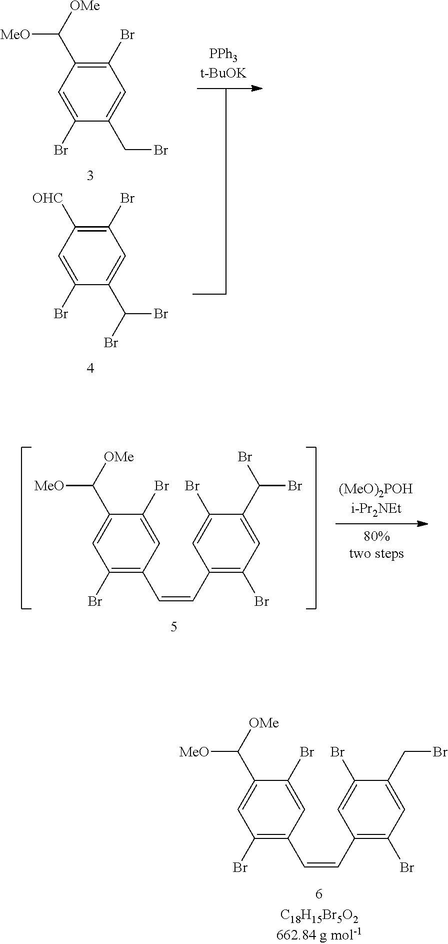

A solution of benzyl bromide (compound 3; 4.03 g, 10.0 mmol, 1 equivalent) and triphenylphosphine (PPh₃; 2.64 g, 10.1 mmol, 1.01 equivalents) in THF/methanol (4:1, 11 mL) was heated under reflux (oil bath at 80° C.) for 3 hours. The obtained transparent solution was cooled to room temperature, and compound 4 (4.40 g, 10.1 mmol, 1.01 equivalent) was added thereto. The mixture was cooled in a cold water bath, and potassium tert-butoxide (t-BuOK; 1.2M in THF, 8.2 mL, 1.0 equivalents) was titrated at such a rate that the yellow-orange color of the ylide intermediate could change between drops. Titration was continued over 30 minutes until the change in color ended. At this point, the reaction was ended, and compound 5 was isolated at a Z/E ratio of 95:5.

Z-5: ¹H NMR (600 MHz, CDCl₃) δ 8.20 (s, 1H), 7.84 (s, 1H), 7.14 (s, 1H), 7.06 s, 1H), 6.88 (s, 1H), 6.78 (A part of an AB system, J=12.0, 1H), 6.74 (B part of an AB system, 1=12.0, 1H), 5.47 (s, 1H), 3.35 (s, 6H).

E-5, characteristic signals. ¹H NMR (600 MHz, CDCl₃) δ 8.22 (s, 1H), 7.84 (s, 1H), 7.77 (s, 1H), 7.30 (A part of an AB system, J=16.0, 1H), 7.27 (B part of an AB system, J=16.0, 1H), 6.97 (s, 1H), 5.52 (s, 1H), 3.40 (s, 6H).

Without isolating compound 5, the reaction product containing compound 5 was further stirred at room temperature for 30 minutes before N,N-diisopropylethylamine (DIPEA; 2.20 mL, 12.6 mmol, 1.26 equivalents) and dimethyl phosphite (1.10 mL, 12.0 mmol, 1.20 equivalents, a slight amount of heat was produced when added) were added thereto. After stirring at room temperature for 1 hour, TLC (n-hexane/acetone, 9:1) confirmed that no starting materials remained and that the materials were completely converted to a dibromo intermediate (compound 5). The mixture was poured on a silica gel pad (d=7 cm, h=3 cm) on which the mixture was eluted with n-hexane/ethyl acetate (9:1) (TLC was used to confirm no more generation of products), and the filtrate was concentrated under reduced pressure. After the title product was obtained as a yellow solid (Z/E 95:5) by flash column chromatography (n-hexane/ethyl acetate, 10:0 to 9:1, added as a hot hexane solution), n-hexane (about 50 mL) was boiled to isolate the stereoisomer. The first isolated product was a Z-isomer, which was a white acicular product (4.63 g). The supernatant was evaporated, and the second crystallization was performed, thereby obtaining an E-isomer (0.249 g) as yellow droplets on the wall of the flask. The supernatant was separated again (the crystallization process was sufficiently slow to allow for the operation), and additional Z-isomer (0.65 g) was obtained as the third isolated product. The total yield was as follows: Z-6: 80% and E-6: 4%.

Z-6: tiny white needles, m.p.: 91.5-92.5° C. (n-hexane). ¹H NMR (600 MHz, CDCl₃) δ 7.84 (s, 1H), 7.67 (s, 1H), 7.15 (s, 1H), 7.14 (s, 1H), 6.75 (A part of an AB system, J=12.0, 1H), 6.74 (B part of an AB system, J=12.0, 1H), 5.47 (s, 1H), 4.48 (s, 2H), 3.35 (s, 6H). ¹³C NMR (100 MHz, CDCl₃) δ 138.0, 137.92, 137.91, 137.8, 134.9, 134.5, 134.1, 132.6, 130.9, 130.1, 122.9, 122.7, 122.6, 121.0, 101.5, 53.4 (2C), 31.8. HRMS of the triphenylphosphine adduct; m/z [C₃₆H₃₀(⁷⁹Br)₂(⁸¹Br)₂O₂P]⁺ calcd.: 844.8671, found: 844.865.

E-6: yellow clusters, m.p.: 137-139° C. (n-hexane). ¹H NMR (400 MHz, CDCl₃) δ 7.86 (s, 1H), 7.84 (s, 1H), 7.83 (s, 1H), 7.69 (s, 1H), 7.29 (A part of an AB system, J=16.1, 1H), 7.27 (B part of an AB system, J=16.1, 1H), 5.52 (s, 1H), 4.55 (s, 2H), 3.40 (s, 6H). ¹³C NMR (100 MHz, CDCl₃) δ 138.2 (3C), 137.9, 135.1, 132.9, 131.3, 131.0, 130.3, 129.5, 123.5, 123.1, 123.0, 121.8, 101.9, 53.8 (2C), 31.9.

### Example 2: (Z,Z)-2,5-dibromo-4-(2,5-dibromo-4-(2,5-dibromo-4-(bromomethyl) styryl) styryl)benz aldehyde dimethyl acetal (Compound 8)

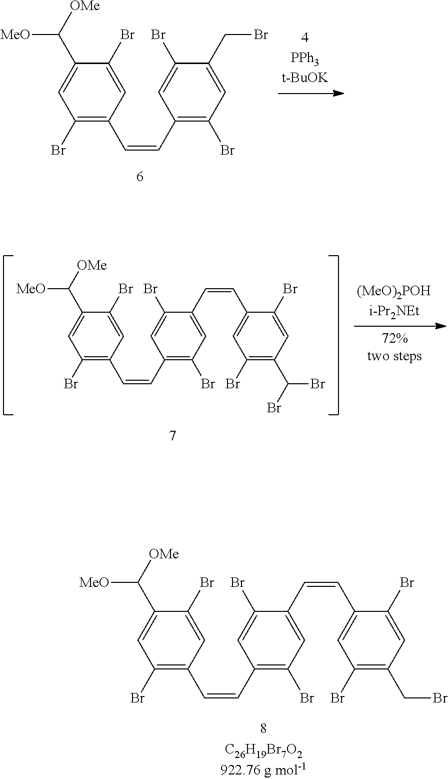

A solution of benzyl bromide (compound 6; 11.1 g, 16.8 mmol, 1 equivalent) and triphenylphosphine (PPh₃; 4.57 g, 17.4 mmol, 1.04 equivalents) in THF/methanol (9:1, 20 mL) was heated under reflux (an oil bath at 80° C.) for 3.5 hours. The obtained transparent, pale-yellow solution was cooled to room temperature, and THF (120 mL) was added thereto. Subsequently, a few droplets of potassium tert-butoxide (t-BuOK; 1.0M in THF) was added while the orange color indicating the generation of an ylide intermediate was persistent. Compound 4 (7.45 g, 17.1 mmol, 1.02 equivalents) was added thereto, and the obtained transparent solution was cooled in an ice water bath. Potassium tert-butoxide (t-BuOK; 1.0M in THF, 16.5 mL, 16.5 mmol, 0.97 equivalents) was then dropped thereto at such a rate that the color of the ylide intermediate changed from its yellow-orange color between droplets. Titration was continued over 10 minutes until the change in color was not observed. Accompanying this addition, a dibromo intermediate (compound 7) was partly precipitated. Before N,N-diisopropylethylamine (DIPEA; 4.00 mL, 23.0 mmol, 1.37 equivalents) and dimethyl phosphite (2.00 mL, 21.8 mmol, 1.30 equivalents) were added, the reaction product containing compound 7 was further stirred at the same temperature for 15 minutes. After stirring at room temperature for 3 hours, most of the initial precipitates returned to the solution. TLC (n-hexane/acetone, 5:1) confirmed that no starting materials remained, and that the materials were completely converted into a dibromo intermediate (compound 7). About 30 g of silica gel was added to the reaction mixture, and the solvent was evaporated under reduced pressure. The result was purified by flash column chromatography (n-hexane/CH₂Cl₂, 10:0 to 1:1, fast gradient), thereby obtaining compound 8 as a yellow solid (14.5 g, 93%, Z,Z/Z,E: 95:5).

Subsequently, the same procedure was performed using compound 6 (5.00 g, 7.54 mmol) as a starting material, and recrystallization from CHCl₃/methanol was further performed, thereby obtaining a pure Z,Z-isomer (5.02 g, 72%). Methanol (50 mL) was added to a hot solution of compound 7 (Z,Z/Z,E, 95:5) in CHCl₃ (about 50 mL), and the solution was boiled to the point at which the solution was decreased to about half the initial volume. After cooling to room temperature, yellow amorphous precipitates were rapidly formed, and the precipitates were separated from the solution through filter paper. After the precipitates were allowed to stand, Z,Z-8 was slowly crystallized from the filtrate as a pale yellow acicular product (filtration can be suitably performed due to the slow crystallization process). When acetone was used instead of CHCl₃, an expected compound was obtained at an equivalent yield and purity.

Z,Z-8, pale yellow needles, m.p.: 142-143V (CHCl₃/MeOH), 140-142° C. (acetone/MeOH). ¹H NMR (600 MHz, CDCl₃) δ 7.84 (s, 1H), 7.68 (s, 1H), 7.23 (s, 1H), 7.22 (s, 1H), 7.17 (s, 1H), 7.15 (s, 1H), 6.71 (A part of an AB system, J=12.0, 1H), 6.70 (B part of an AB system, J=12.0, 1H), 6.69 (app s, weak symmetrical side signals indicate a very strongly coupled AB system J≈12, 2H), 5.49 (s, 1H), 4.50 (s, 2H), 3.35 (s, 6H). ¹³C NMR (150 MHz, CDCl₃) δ 138.1, 138.0, 137.9, 137.7, 137.0, 136.8, 134.9, 134.5, 134.17, 134.15, 134.0, 132.7, 130.7, 130.39, 130.37, 129.9, 122.8, 122.7, 122.6, 122.3, 122.1, 121.1, 101.5, 53.4 (2C), 31.8. HRMS; of the triphenylphosphine adduct, m/z [C₄₄H₃₄(⁷⁹Br)₃(⁸¹Br)₃O₂P]⁺, calcd.: 1104.7330, found: 1104.730.

Characteristic signals for Z,E-8, NMR (600 MHz, CDCl₃) δ 7.87 (s, 1H), 5.48 (s, 1H), 4.53 (s, 2H), 3.39 (s, 6H)_{;}

### Example 3: (Z,Z)-2,5-dibromo-4-(2,5-dibromo-4-(2,5-dibromo-4-carbaldehyde-styryl)styryl)benzyl triphenyl phosphonium hexafluorophosphate (Compound 9)

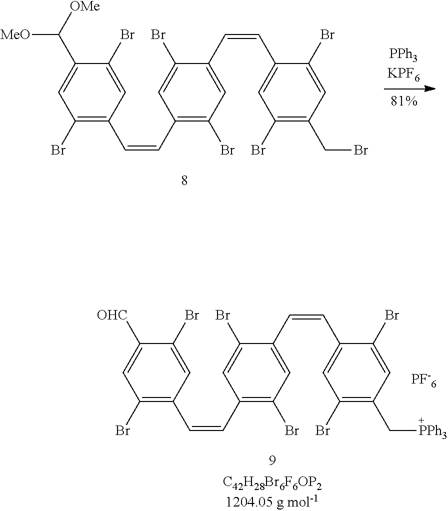

A mixture of partly soluble benzyl bromide (compound 8; 15.8 g, 17.1 mmol) and triphenylphosphine (PPh₃; 4.56 g, 17.4 mmol) in THF (120 mL) and methanol (30 mL) was heated under reflux for 5 hours (an oil bath at 80° C.). The solvent was removed under reduced pressure, and the obtained solid was washed with diethyl ether (Et₂O; 80 mL×3), thereby obtaining phosphonium bromide as a white solid. Acetone (120 mL) and hydrochloric acid (4M, 30 mL) were added to the obtained solid, and the mixture was stirred for 2 hours. Subsequently, acetone was removed under reduced pressure, and CH₂Cl₂ (100 mL) and water (50 mL) were added thereto. The mixture was carefully neutralized with a saturated NaHCO₃ aqueous solution until the aqueous phase reached a neutral pH (about 110 mL). The layers were separated, and water (50 mL) and KPF₆ (7.13 g, 38.7 mmol) were added to the organic phase, followed by intensively stirring the mixture for 3 minutes. The mixture was extracted with CH₂Cl₂ (3 times), and the organic layer was washed with brine and dried over Na₂SO₄, followed by concentration under reduced pressure. Subsequently, the obtained yellow solid was dissolved in hot CHCl₃ (120 mL), and ethyl acetate (120 mL) was added to cool the result, thereby obtaining compound 9 as a bright shiny yellow crystal. NMR confirmed the formation of a 1:1 cocrystal containing ethyl acetate (compound 9.AcOEt) (20.3 g, 92%). Regardless of the presence of ethyl acetate, a Wittig reaction afterward cannot be avoided. However, after dissolution and evaporation were continuously performed in CHCl₃, an amorphous solid free from the solvent used in ¹³C NMR analysis was obtained.

Z,Z-9.AcOEt: fine yellow platelets, m.p.: 136-140° C. (CHCl₃/AcOEt), turned into a sticky wax. ¹H NMR (600 MHz, CDCl₃) δ 10.21 (s, 1H), 8.11 (s, 1H), 7.85 (t with fine structure, J=8, 3H), 7.68 (td, J=8, 4, 6H), 7.55 (dd with fine structure, J=13, 8, 6H), 7.28 (s, 1H), 7.25 (d, J=27, 1H), 7.20 (s, 1H), 7.18 (s, 1H), 7.10 (s, 1H), 6.81 (A part of an AB system, J=12.0, 1H), 6.77 (B part of an AB system, J=12.0, 1H), 6.70 (A part of an ABX system, J=12.0, 0, 1H), 6.65 (B part of an ABX system, J=12.0, 2, 1H), 4.76 (d, J=14, 2H). ¹³C NMR (150 MHz, CDCl₃) δ 189.8, 143.3, 138.8 (d, J=4), 137.4, 136.7, 136.2 (d, J=5), 135.7 (d, J=3, 3C), 135.0, 134.10, 134.02 (d, J=3), 133.92 (d, J=10, 6C), 133.88, 133.80, 133.3, 131.8, 130.9 (d, J=1), 130.5 (d, J=13, 6C), 130.0, 129.7 (d, J=2), 128.2 (d, J=9), 125.0 (d, J=6), 124.6, 123.5, 123.3 (d, J=4), 122.3, 122.1, 116.0 (d, J=86, 3C), 30.3 (d, J=51).* ³¹P NMR (240 MHz, CDCl₃) 22.3, −143.7 (sept, J=714). HRMS; m/z [C₄₂H₂₈(⁷⁹Br)₃(⁸¹Br)₃OP]⁺ calcd.: 1058.6912, found: 1058.691.

In ¹³C NMR analysis, couplings with the ³¹P nucleus were unambiguously attributed by recording spectra at two different frequencies (¹³C 100 and 150 MHz, respectively).

### Example 4: All-Z-[6]cyclopara-2,5-dibromophenyl ethynylene (Cyclic Compound 10)

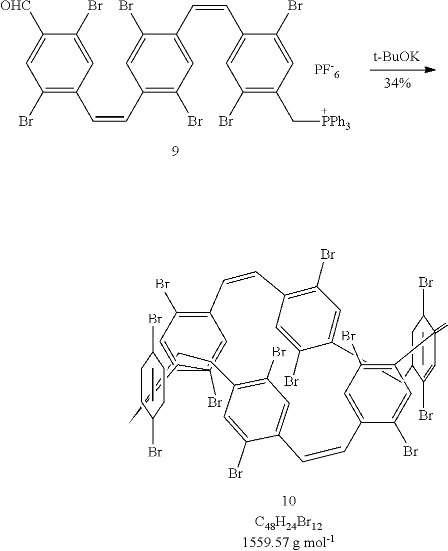

Potassium tert-butoxide (t-BuOK; 1.0M in THF, 12.5 mL, 12.5 mmol) was added to a solution of compound 9.AcOEt (13.3 g, 10.3 mmol) in CH₂Cl₂ (500 mL) over 3 minutes at 0° C. The obtained dark-red non-homogeneous mixture was further stirred at room temperature for 80 minutes, and saturated NH₄Cl (200 mL) was added thereto to neutralize the excessive base. The red color of the mixture gradually disappeared when the mixture was shaken, and the obtained non-homogeneous yellow mixture was filtered. The filtrate was extracted with CH₂Cl₂, and the combined organic layer was washed with brine, dried over Na₂SO₄, and filtered, followed by concentration under reduced pressure. The crude product was purified by flash column chromatography (n-hexane/CH₂Cl₂, 95:5 to 6:4) to obtain 4.12 g of an yellow powder, followed by boiling of toluene (about 80 mL) to perform recrystallization.*

The first product isolated by recrystallization was observed as a cocrystal of the pale yellow crystal of cyclic compound 10 with bimolecular toluene (cyclic compound 10.(tol)₂; 3.02 g), as confirmed in NMR and X-ray crystallographic analysis. From the second isolated product, 0.24 g of the material was further obtained (total yield: 36%). A crystal suitable for X-ray crystallographic analysis was also obtained by this method.
When the solid obtained after chromatography was dissolved in hot toluene, a supersaturated solution was obtained. A small amount of cyclic compound 10 was rapidly precipitated as a solvent-free microscopic square-shaped colorless crystal when measured by ¹H NMR (melting point: 303-309° C.).

10.(toluene)₂, white to pale yellow needles (toluene), 309-312° C. ¹H NMR (400 MHz, CDCl₃) δ 7.29 (bs, 12H), 6.68 (s, 12H). ¹H NMR (600 MHz, CD₂Cl₂) δ 7.2 9 (bs, 12H), 6.71 (s, 12H). ¹³C NMR (100 MHz, CDCl₃) δ 137.1 (12C), 134.4 (12 C), 130.1 (12C), 122.0 (12C). HRMS; m/z. [C₄₈H₂₄(⁷⁹Br)₆(⁸¹Br)₆(⁸¹Br)₆]⁺⁺ calcd.: 1559.1951, found: 1559.193.

### Example 5: [12]Isocyclophenacene (Compound 11; [12]ICP)

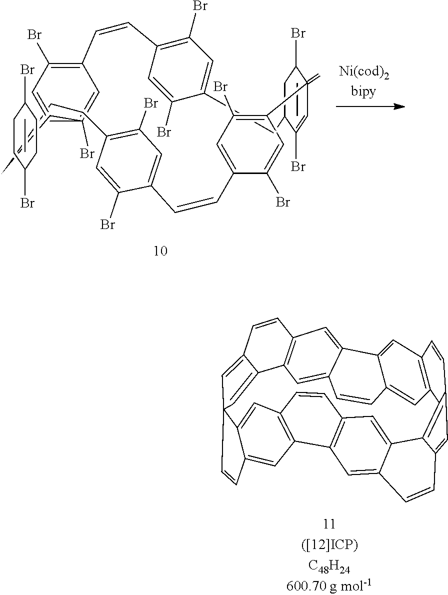

Bis(1,5-cyclooctadiene)nickel(0) (Ni(cod)₂; 1.65 g, 6.00 mmol, 12.0 equivalents) was placed in a three-necked round-bottom flask equipped with a thermometer and dropping funnel beforehand in an argon atmosphere in a glove box. Subsequently, 2,2′-bipyridyl (0.939 g, 6.01 mmol, 12.0 equivalents) and dry dimethylformamide (DMF; 50 mL) were added thereto. The mixture was stirred at room temperature for 30 minutes until a dark purple solution was obtained. This solution was set in an oil bath at 75° C. and stirred for 5 minutes until the temperature of the solution stabilized at 70° C. Subsequently, a solution of cyclic compound 10-(tol)₂ (0.877 g, 0.500 mmol) in hot toluene (50 mL, sufficiently heated to dissolve all of the substances) was added thereto at one time through the dropping funnel. This mixture was then stirred at the same temperature for 15 minutes, and then withdrawn from the oil bath. The hot mixture was then poured into a mixture of toluene (200 mL) and a saturated NH₄Cl aqueous solution (200 mL). The flask was rinsed with toluene, and the mixture was intensively stirred for 10 minutes. The obtained suspension was filtered through filter paper and a collected orange-colored solid was sufficiently washed with toluene. The layers of the filtrate were separated, and the aqueous phase was extracted with toluene once again. Subsequently, the combined organic layer was washed with a saturated NH₄Cl aqueous solution twice and with brine 3 times, dried over Na₂SO₄, and filtered, followed by concentration under vacuum. The obtained, solid orange-colored residue was washed with n-hexane to remove impurities and dissolved in CH₂Cl₂ again, followed by filtration through a short pad of silica gel (d=3 cm, h=2 cm) using CH₂Cl₂ as an eluent (about 100 mL). N,N-dimethylacetamide (DMA; 2 mL) and CHCl₃ (50 mL) were added to the filtrate, and the obtained transparent orange-colored solution was concentrated with a rotary distiller (40° C., 400 mbar) until the solvent was distilled and disappeared. After cooling, the title compound was crystallized from the remaining DMA/CHCl₃ as tiny rectangle red crystals. A few milliliters of CHCl₃ was further added, and the mixture was allowed to stand for 2 hours to complete the crystallization process. The obtained crystals were filtered (a supernatant containing crystals in a suspension can be treated with a Pasteur pipette) and washed with CHCl₃ a few times, followed by drying under high vacuum, thereby obtaining [12]ICP (carbon nanobelt 11.(CHCl₃)₃) crystallized with trimolecular CHCl₃ (4 mg, 1%), as shown in NMR and X-ray structure diffraction.

On a small scale, a solid obtained by filtration through silica gel was dissolved in CHCl₃, and a transparent solution was first formed. A trace amount of red crystals was precipitated in a substantially quantitative manner within a few minutes. Most of the crystals of carbon nanobelt 11.(CHCl₃)₃ did not dissolve in CH₂Cl₂, but the crystals were reasonably soluble in CS₂, N,N-dimethylacetamide (DMA), or 1,3-dimethyl-3,4,5,6-tetrahydro-2-pyrimidinone (DMPU). Crystals suitable for X-ray structure diffraction were grown by slowly diffusing a mixture of cyclohexane and CHCl₃ (cyclohexane/CHCl₃, 3:1) in a solution of carbon nanobelt 11.(CHCl₃)₃ in DMPU.

11.(CHCl₃)₃ red-orange rectangular crystals, m.p.: >350° C. (from DMPU/CHCl₃). ¹H NMR (600 MHz, CD₂Cl₂) δ 8.32 (s, 12H1), 7.55 (s, 12H2). ¹³C NMR (150 MHz, CD₂Cl₂) δ 132.7 (C3), 128.1 (C4), 126.7 (C2), 122.8 (C1). HRMS; m/z [C₄₈H₂₄]⁺⁺ calcd.: 600.1872, found: 600.1857.

The ¹³C NMR signal was identified based on HSQC and HMBC NMR experiments. J³ C—H exhibits a stronger correlation in HMBC than J² C—H. However, a weak correlation observed in H2 is assigned to J²_{H2} and J³_{H2′} of C3, not J³_{H2} and J⁴_{H2′} of C4, and follows theoretically predicted relative chemical shift values.

### Synthesis of [16]Isocyclophenacene ([16]ICP) and [24]isocyclophenacene ([24]ICP)

### Example 6:(Z,Z,Z)-2,5-dibromo-4-(2,5-dibromo-4-(2,5-dibromo-4-(2,5-dibromo-4-(bromomethyl)styryl)styryl)styryl)-benzaldehyde dimethyl acetal (Compound 12)

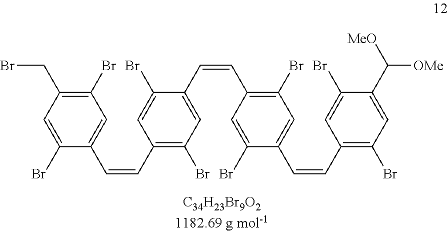 [SMILES: COC(OC)c1cc(Br)c(/C=C\c2cc(Br)c(/C=C\c3cc(Br)c(/C=C\c4cc(Br)c(CBr)cc4Br)cc3Br)cc2Br)cc1Br]

A solution of a benzyl bromide trimer (compound 8; 2.99 g, 3.24 mmol, 1 equivalent) and triphenylphosphine (PPh₃; 0.900 g, 3.43 mmol, 1.06 equivalents) in THF/methanol (6:1, 17.5 mL) was heated under reflux (an oil bath at 80° C.) for 3 hours. The obtained yellow solution was cooled to room temperature, and compound 4 (1.45 g, 3.33 mmol, 1.03 equivalents) was added thereto, followed by adding THF (20 mL). The mixture was cooled in a cold water bath to maintain a temperature around room temperature during the reaction, and potassium tert-butoxide (t-BuOK; 1.10M in THF, 2.93 mL, 1.00 equivalent) was added dropwise over 10 minutes in total at such a rate that the red color of the ylide intermediate faded away between droplets. t-BuOK was continuously added until the change in color due to t-BuOK was not observed. A dibromomethyl intermediate was gradually precipitated in line with the addition of t-BuOK. Before N,N-diisopropylethylamine (0.800 mL, 4.59 mmol, 1.42 equivalents) and dimethyl phosphite (0.400 mL, 4.36 mmol, 1.35 equivalents) were added, the reaction product was further stirred at room temperature for 20 minutes, and then cooled in an ice bath. After stirring at the same temperature for 5 minutes, the ice bath was removed, and stirring was continued for 4 hours. During this operation, the dibromomethyl intermediate was confirmed to have been completely consumed by TLC (hexane/acetone=4:1). Subsequently, the obtained non-homogeneous mixture was concentrated under reduced pressure, and the obtained yellow semi-solid was dissolved in hot CHCl₃ again and poured onto a short silica gel pad (d=7 cm, h=4 cm) for thorough elution with CHCl₃ (300 mL). Methanol (MeCH; 100 mL) was added to the obtained filtrate, and the solution was concentrated under reduced pressure. At a point at which about 50 mL of the solution remained, the desired compound was precipitated as a yellow solid. The solid was collected by filtration, washed with methanol (MeCH) twice, and then dried under high vacuum, thereby obtaining the target compound 12 as powdery dust (2.68 g, 70%).

### Microscopic Pale-Yellow Powder

¹H NMR (400 MHz, CDCl₃) δ 7.85 (s, 1H), 7.69 (s, 1H), 7.25 (s, 1H), 7.25 (s, 2H), 7.23 (s, 1H), 7.18 (s, 1H), 7.16 (s, 1H), 6.72 (s, 2H), 6.72 (A part of an AB system, J=12, 1H), 6.71 (B part of an AB system, J=12, 1H), 6.67 (s, 2H), 5.49 (s, 1H), 4.51 (s, 2H), 3.36 (s, 6H).

¹³C NMR (100 MHz, CDCl₃) δ 138.3, 138.2, 138.1, 137.8, 137.2, 137.11, 137.06, 137.0, 135.1, 134.7, 134.36, 134.34, 134.30, 134.25 (2C), 132.9, 130.9, 130.6, 13 0.5, 130.4, 130.3, 130.0, 122.91, 122.87, 122.8, 122.47, 122.43, 122.35, 122.26, 1 21.2, 101.6, 53.5 (2C), 32.0.

HRMS (MALDI-TOF, DCTB matrix, NaI) of the triphenylphosphine adduct, m/z [Cs₂H₃₈(⁷⁹Br)₄(⁸²Br)₄O₂P]⁺ calcd.: 1364.5990. found: 1364.596.

### Example 7:(Z,Z,Z)-2,5-dibromo-4-(2,5-dibromo-4-(2,5-dibromo-4-(2,5-dibromo-4-carbaldehyde-styryl)-styryl)-styryl)benzyl triphenyl phosphonium hexafluorophosphate (compound 13)

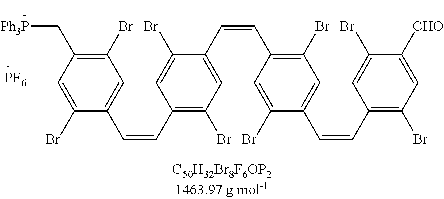

A mixture of partially soluble benzyl bromide (compound 12; 0.695 g, 0.588 mmol, 1 equivalent) and triphenylphosphine (PPh₃; 0.160 g, 0.611 mmol, 1.04 equivalents) in THF/methanol (4:1, 2.5 mL) was heated under reflux for 6 hours (an oil bath at 80° C.). The obtained yellow suspension was cooled to room temperature and concentrated under reduced pressure, thereby obtaining a yellow solid. Acetone (15 mL) and hydrochloric acid (4M, 5 mL) were added to the obtained solid. The obtained heterogeneous yellow mixture was stirred for 9 hours. Subsequently, acetone was evaporated under vacuum, and the mixture was diluted with CHCl₃. The acid was neutralized with a saturated NaHCO₃ aqueous solution, and the layers were separated, followed by extracting the aqueous phase with CHCl₃ again. The organic layer combined again was thoroughly shaken with a KPF₆ (0.25 g in 30 mL of water, about 2 equivalents) aqueous solution. The aqueous layer was discarded, and the organic layer was washed with another KPF₆ aqueous solution (0.1 g in 20 mL of water) again to ensure complete anion metathesis. The organic phase was separated and dried over Na₂SO₄, followed by concentration under vacuum. Subsequently, the obtained yellow solid was dissolved in hot CHCl₃, and an equivalent amount of ethyl acetate (AcOEt) was added thereto, followed by cooling, thereby obtaining microscopic shiny yellow crystals. The crystals were then filtered, and washed with ethyl acetate (AcOEt), followed by drying under high vacuum. The formation of a 1:1 cocrystal containing ethyl acetate (compound 13.AcOEt) (0.725 g, 80%) was confirmed by NMR. Dissolution and evaporation were continuously performed in CHCl₃, thereby obtaining an amorphous solid that is free from the solvent used in ¹³C NMR analysis.

Microscopic Yellowish Orange Platelets:

m.p.: 137-145° C. (CHCl₃/AcOEt), wax ¹H NMR (600 MHz, CDCl₃) δ 10.21 (s, 1H), 8.11 (s, 1H), 7.85 (t with fine structure, J=8, 3H), 7.68 (td, J=8, 4, 6H), 7.55 (dd with fine structure. J=13, 8, 6H), 7.32 (s, 1H), 7.26 (s, 1H), 7.24 (s, 1H), 7.23 (s, 1H), 7.18 (s, 2H), 7.11 (s, 1H), 6.81 (A part of an AB system, J=12.0, 1H), 6.76 (8 part of an AB system, J=12.0, 1H), 6.71 (A part of an ABX system, J=12.0, 0, 1H), 6.70 (s, 2H), 6.65 (B part of an ABX system, J=12.0, 2, 1H), 4.73 (d, J=14, 2H). ¹³C NMR (150 MHz, CDCl³, 60° C.) δ 189.6, 143.5, 139.2 (d, J=4), 137.6, 137, 4 (2C), 137.0, 136.4 (d, J=5), 135.8 (d, J=3, 3C), 135.3, 134.35, 134.30 (d, J=2), 134.26 (2C), 134.13 (d, J=10, 6C), 134.07, 133.9, 133.7, 132.1, 131.2 (d, J=1), 130.6 (d, J=13, 6C), 130.5, 130.3, 130.0, 129.8 (d, J=2), 128.4 (d, J=9), 125.1 (d, J=7), 124.6, 123.6, 123.5 (d, J=4), 122.5, 122.4, 122.33, 122.31, 116.5 (d, J=86, 3C). 30.7 (d, J=51).

¹³P NMR (240 MHz, CDCl³, 60° C.) δ 22.4, −143.7 (sept. J=714).

HRMS (MALDI-TOF, DCTB matrix, NaI), [C₅₀H₃₂(⁷⁹Br)₄(⁸²Br)₄OP]⁺ m/z calcd.: 1318, 5571, found: 1318.556.

### Example 8: Oligomerization (Synthesis of Cyclic Compound 14)

A solution of potassium tert-butoxide (t-BuOK; 0.23M in THF, 0.3 mL, 0.25 equivalents) was added dropwise to a solution of a phosphonium salt (compound 13.AcOEt; 0.446 g, 0.28 mmol, 1 equivalent) in CH₂Cl₂ (1.5 mL) at room temperature. Yellow precipitates were readily formed by the addition of t-BuOK. After completion of the addition of t-BuOK, the mixture was further stirred for 5 minutes, and microscopic precipitates were collected by filtration (filter paper). The collected solid was washed with CH₂Cl₂ twice, and then dried under vacuum. The filtrate containing the remaining starting materials and the washing solution were concentrated under reduced pressure. The residue was dissolved in another compound 13.AcOEt (0.220 g, 0.14 mmol) dissolved in CH₂Cl₂ (1.5 mL), and treated with t-BuOK (0.23M in THF, 0.3 mL, 0.25 equivalents), followed by collection in the same manner as described above, thereby obtaining the second precipitates. The filtrate was concentrated, and dissolved in another compound 13.AcOEt (0.213 g, 0.13 mmol) dissolved in CH₂Cl₂ (1.5 mL), followed by the third treatment of adding t-BuOK (0.23M in THF, 0.3 mL, 0.25 equivalents). The three collected products were combined and dried under high vacuum, thereby obtaining 0.517 g of a crude oligomer. A ¹H NMR spectrum was recorded in high-temperature C₂D₂Cl₄, and a signal that was characteristic for a small amount of a cyclic tetramer (cyclic compound 14), together with aldehyde and a benzyl phosphonium proton of the linear oligomer, was observed. The presence of octamer and dodecamer phosphonium intermediates was also confirmed by MALDI-TOF analysis (DCTB matrix).

### (Z,Z,Z,E)-[4]cyclo-2,5-dibromo-para-phenylene-ethenylene (cyclic compound Z,Z,Z,E-14)

A pure sample of cyclic compound Z,Z,Z,E-14 was obtained by crystallization due to evaporation of CH₂Cl₂. A single crystal suitable for an XRD experiment was grown through slow diffusion in a solution of n-hexane in CHCl₃ (prepared by subjecting the material to ultrasound treatment in boiling CHCl₃).

¹H NMR (600 MHz, CDCl₃) δ 7.89 (s, 2H), 7.39 (s, 2H), 7.08 (s, 2H), 6.96 (s, 2H), 6.95 (d, 1=11, 2H), 6.71 (d, J=11, 2H), 6.39 (s, 2H), 6.22 (s, 2H).

13C NMR (100 MHz, CS₂/CDCl₃ 4: 1) δ 137.1, 133.6, 129.5, 121.7.

### (Z,Z,Z,Z)-[4]cyclo-2,5-dibromo-para-phenylene-ethenylene (cyclic compound Z,Z,Z,Z-14)

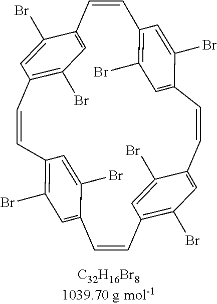 [SMILES: Brc1cc2c(Br)cc1/C=C\c1cc(Br)c(cc1Br)/C=C\c1cc(Br)c(cc1Br)/C=C\c1cc(Br)c(cc1Br)/C=C\2]

A pure sample of cyclic compound Z,Z,Z,Z-14 was obtained by crystallization due to evaporation of CH₂Cl₂. A single crystal suitable for an XRD experiment was grown through slow diffusion in a solution of n-hexane in CH₂Cl₂/CS₂.

¹H NMR (600 MHz, CDCl₃) δ 7.37 (s, 8H), 6.66 (s, 8H).

¹³C NMR (150 MHz, CS₂/CDCl₃ 4: 1) δ 139.5, 138.5, 138.3, 136.7, 135.4, 134.8, 134.6, 133.9, 132.4, 132.0, 129.1, 127.5, 123.1, 123.0, 121.8, 121.4.

### Example 9: Macroring Formation (Synthesis of Cyclic Compound 14, Cyclic Compound 15, and Cyclic Compound 16)

The oligomer mixture obtained in Example 8 (0.517 g) was dissolved in hot C₂H₂Cl₄ (100 mL, >100° C.), and the solid aggregates were broken down by ultrasound treatment. The obtained opaque yellow solution was magnetically stirred in an oil bath at 135° C., and then maintained at room temperature in a cold water bath while a KOH non-homogeneous mixture (10 g) in CH₂Cl₂/ethanol (9:1, 100 mL) was slowly added through an inserted cannula at 1 or 2 droplets per second. After 1.5 hours, the addition was completed, and water was added to dissolve the inorganic salt. Subsequently, the mixture was extracted with CH₂Cl₂ one time and with CHCl₃ one time. The combined organic layer was then continuously washed with 1N hydrochloric acid and brine (twice), dried over Na₂SO₄, and filtered, followed by concentration under reduced pressure. The brown residue was placed in a flash chromatograph (hexane/CHCl₃ 10:0 to 75:25) as a CS₂ solution (the residue was dissolved by ultrasound treatment because not all of the residue was soluble), and purified in the following elution order: a cyclic tetramer (cyclic compound 14), which was a mixture of a Z,Z,Z,E isomer and a Z,Z,Z,Z isomer (51 mg), followed by a pure Z,Z,Z,Z isomer (17 mg), an all-Z cyclic octamer (cyclic compound 15) in a mixture containing another compound that was tentatively attributable to a Z,Z,Z,Z,Z,Z,Z,E isomer (82 mg, Z,Z,Z,Z,Z,Z,Z,Z/Z,Z,Z,Z,Z,Z,Z,E=3:1 according to the integration of a ¹H NMR spectrum), and a cyclic dodecamer (cyclic compound 16) in a mixture containing an identified by-product (32 mg).

[8]Cyclo-2,5-dibromo-para-phenylene-Z-ethenylene (cyclic compound 15)

A pure sample of Z,Z,Z,Z,Z,Z,Z,Z-15 (cyclic compound 15) was obtained by recrystallization from boiling toluene. However, due to the difficulty in resolubilizing the purified compound, the synthesis of [16]isocyclophenacene (compound 17; [16]ICP) was performed directly from an amorphous mixture obtained after chromatography for better convenience.

¹H NMR (600 MHz, CS₂/CD₂Cl₂ 5: 1) δ 7.28 (s, 16H), 6.68 (s, 16H).

¹³C NMR (150 MHz, CS₂/CD₂Cl₂ 5: 1) δ 137.8, 134.7 130.9, 123.1.

HRMS (MALDI-TOF, DCTB matrix, NaI), m/z [C₆₄H₃₂(⁷⁹Br)₈(³¹Br)₈]⁺ calcd.: 2078.926 9, found: 2078.9420.

[12]Cyclo-2,5-dibromo-para-phenylene-Z-ethenylene (cyclic compound 16)

For cyclic compound 16, [24]isocyclophenacene (compound 18; [24]ICP) was synthesized directly from an amorphous mixture obtained after chromatography, as with cyclic compound 15.

HRMS (MALDI-TOF, DCTB matrix, NaI), m/z [C₉₆H₄₈(⁷⁹Br)₁₂(⁸¹Br)₁₂]⁺ calcd.: 3118.3 906, found: 3118.3975.

### Example 10: [16]Isocyclophenacene (compound 17; [16]ICP)

Bis(1,5-cyclooctadiene)nickel(0) (Ni(cod)₂; 0.31 g, 1.13 mmol, 16 equivalents) and 5,5′-dimethoxycarbonyl-2,2′-bipyridine (0.31 g, 1.14 mmol, 16 equivalents) were placed in a round-bottom flask in an argon atmosphere in a glove box. Dimethylacetamide (DMA; 10 mL) was further added to the round-bottom flask, and the mixture was stirred at room temperature for 20 minutes, thereby obtaining a dark-green solution composite (not all reagents were dissolved). The round-bottom flask was then placed in an oil bath at 80° C. After stirring at this temperature for 5 minutes, all of the reagents were dissolved, and a cyclic octamer (cyclic compound 15; 0.146 g, 70.2 μmol, obtained by chromatography) in hot toluene (10 mL, a temperature right below the boiling point) was quickly added thereto through a syringe. After the addition of the cyclic octamer, the mixture was stirred at the same temperature for 30 minutes, and then cooled to room temperature. Subsequently, the non-homogeneous, dark-colored mixture was diluted with toluene, washed with brine four times, dried over Na₂SO₄, and filtered, followed by concentration in vacuum. The resulting crude yellow-green product was dissolved in CS₂ and placed in silica gel. The result was subjected to flash column chromatography (FC; hexane/CH₂Cl₂=1:1) to obtain the desired [16]isocyclophenacene (compound 17; [16]ICP) as a yellow solid (4 mg, 7%). A sample having a purity of over 95% in HPLC (green fluorescence under UV light) was obtained by performing preparative thin-layer chromatography using CCl₄ as an eluent.

¹H NMR (600 MHz, CD₂Cl₂) δ 8.48 (s, 16H), 7.58 (s, 16H).

NMR (150 MHz, CD₂Cl₂) δ 132.3, 129.2, 127.2, 123.1.

FIRMS (MALDI-TOF, DCTB matrix, NaI), m/z [C₆₄H₃₂]⁺ calcd.: 800.2498, found: 80 0.2463.

### Example 11: [24]Isocyclophenacene (compound 18; [24]ICP)

Bis(1,5-cyclooctadiene)nickel(0) (Ni(cod)₂; 83 mg, 0.30 mmol, 24 equivalents) and 5,5′-dimethoxycarbonyl-2,2′-bipyridine (82 mg, 0.30 mmol, 24 equivalents) were placed in a screw-cap tube in an argon atmosphere in a glove box. Dimethylacetamide (DMA; 2.5 mL) was further added to the screw-cap tube, and the mixture was stirred at room temperature for 20 minutes, thereby obtaining a dark-green solution composite (not all of the Ni(cod)₂ was dissolved). The screw-cap tube was then placed in an oil bath at 80° C. The composite was stirred at this temperature for 5 minutes, and all of nickel and ligands were dissolved. A cyclic dodecamer (cyclic compound 16; 39 mg, obtained by flash column chromatography) in toluene (2.5 mL) was then quickly added through a syringe. After the addition of the cyclic dodecamer, the mixture was stirred at the same temperature for 40 minutes, and then cooled to room temperature. Subsequently, the non-homogeneous dark-colored mixture was diluted with toluene, washed with brine four times, dried over Na₂SO₄, and filtered, followed by concentration in vacuum. The residue was then dissolved in CH₂Cl₂ and filtered through a short plug of silica gel (Pasteur pipette), followed by elution with CH₂Cl₂. After the solvent was evaporated, the residue was completely dissolved in CDCl₃. Isopropyl alcohol (i-PrOH; 5 μL, 65 μmol) was then added as an internal standard, and the ¹H NMR spectrum was recorded. From the signal integration, about 0.25 μmol of the desired [24]isocyclophenacene (compound 18; [24]ICP; 2% from cyclic compound 16, 0.1% from compound 13) was confirmed to have formed. A sample having a purity over 90% in HPLC (a pale yellow solid, blue fluorescence under UV light) was obtained by preparative thin-layer chromatography using CCl₄/CH₂Cl₂ as an eluent.

H NMR (600 MHz, CD₂Cl₂) δ 8.90 (s, 24H), 7.79 (s, 24H).

¹³C NMR (150 MHz, CD₂Cl₂) δ 132.0, 129.6, 127.8, 123.2.

HRMS (MALDI-TOF, DCTB matrix, NaI), m/z [C₉₆H₄₈]⁺ calcd.: 1200.3750, found: 1 200.3765.

### Test Example 1: X-Ray Structure Diffraction

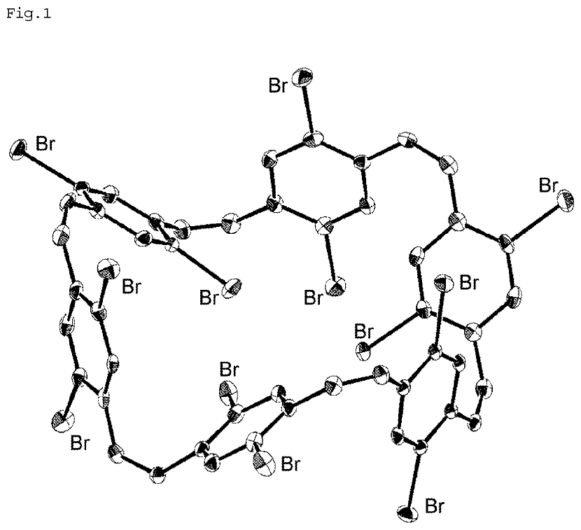

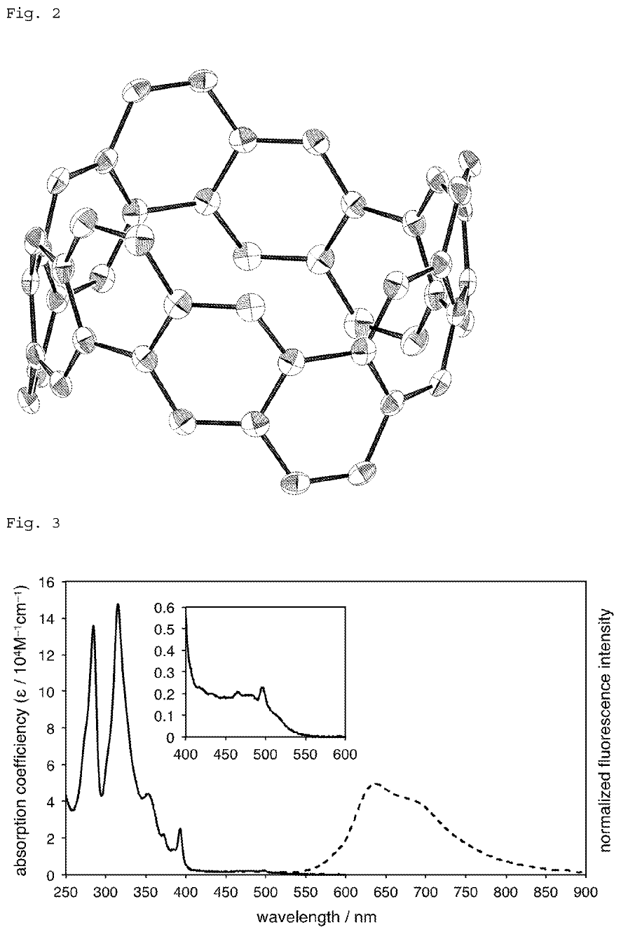

Table 1 illustrates details of crystal data and an overview of strength data acquisition parameters for cyclic compound 10.(tol)₂ and [12]ICP (carbon nanobelt 11.(CHCl₃)₃). FIGS. 1 to 2 illustrate the structure of cyclic compound 10-(tol)₂ and [12]ICP (carbon nanobelt 11.(CHCl₃)₃) determined by X-ray structure diffraction. In both cases, a suitable crystal was mounted on a glass fiber by using mineral oil and transferred to the goniometer of a Rigaku PILATUS diffractometer. Graphitic monochromatic MoKα rays were used. The structure was determined by a direct technique using SIR-97 (J. Appl. Crystallogr., 1999, 32, 115), and normalized by applying a full-matrix least-squares method to F² (SHELXL-2013/4)(Acta Crystallographica Section A, 2008, 64, 112) using the Yadokari-XG program. The strength was normalized in regards to a Lorentz and polarization effect.

TABLE 1

| | | 11 |
|---|---|---|
| | 10 | ([12]ICP) |
| CCDC | C62H40Br12 | C51H24Cl9 |
| formula | | |
| fw | 1743.86 | 955.75 |
| T (K) | 123(2) | 123(2) |
| λ (Å) | 0.71075 | 0.71073 |
| cryst syst | Triclinic | Orthorhombic |
| space group | P-1 | Pnnm |
| a (Å) | 8.8829(8) | 9.8350(2) |
| b (Å) | 15.3724(15) | 13.7023(2) |
| c (Å) | 21.600(2) | 15.3817(3) |
| α (deg) | 93.242(2) | 90 |
| β (deg) | 93.364(3) | 90 |
| γ (deg) | 93.819(3) | 90 |
| V (Å³) | 2932.5(5) | 2072.87(7) |
| Z | 2 | 2 |
| D_{calc} (g/cm³) | 1.975 | 1.531 |
| μ (mw⁻¹) | 8.235 | 0.647 |
| F(000) | 1664 | 966 |
| cryst size (mm) | 0.20 × 0.15 × 0.15 | 0.20 × 0.05 × 0.05 |
| θ range (deg) | 3.062-24.999 | 2.458-24.985 |
| reflns collected | 32673 | 28486 |
| indep reflns/Rᵢₙₜ | 10268/0.0671 | 1903/0.0280 |
| Params | 669 | 184 |
| GOF on F² | 0.995 | 1.089 |
| R₁, wR₂ [l > 2σ(l)] | 0.0399, 0.0958 | 0.0653, 0.1978 |
| R₁, wR₂ (all data) | 0.0585, 0.1046 | 0.0685, 0.2010 |

### Test Example 2: Photophysical Properties

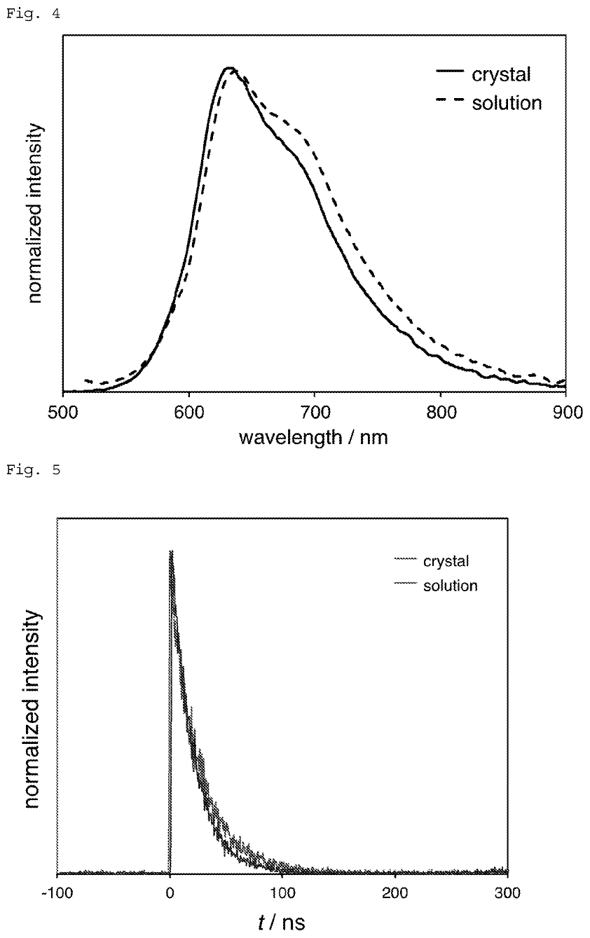

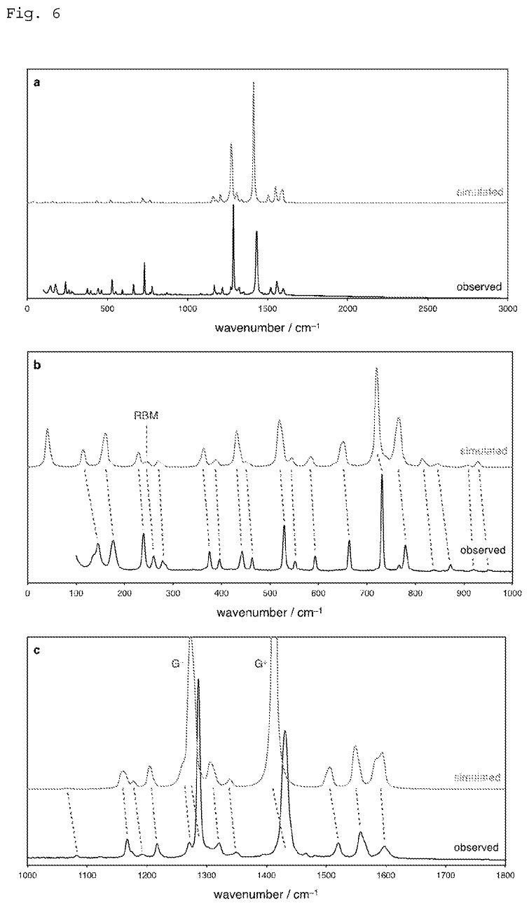

All measurements were performed using a diluted solution of dichloromethane of degasification spectrum grade in a 1-cm-square quartz cell. The UV-vis absorption spectra were recorded with a Shimadzu UV-3510 spectrometer at a resolution of 0.5 nm. The photoluminescence (PL) was measured with a wavelength-turnable optical parametric amplifier system based on Yb: KGW (potassium tungstate) regenerative amplification laser (a pulse duration of 200 fs and a repeating rate of 200 kHz) for the excitation light source. PL spectra and decay dynamics were detected with a monochromator equipped with a charge-coupled device (CCD) camera (Princeton Instruments, ProEM) and an avalanche photodiode (Micro Photon Devices, SPD-050-CTE-N1). PL decay dynamics was measured by synchronizing a detected wavelength and a PL peak wavelength, and each photon arrival time was recorded with a time-correlated single-photon counting board (Becker & Hickl GmbH, SPC-130EM-N1). Absolute fluorescence quantum yields were determined with a Hamamatsu C9920-02 integrating sphere calibration system, with excitation at 390 nm for [12]ICP. FIGS. 3 to 5 illustrate the results.

### Test Example 3: Raman Spectrum

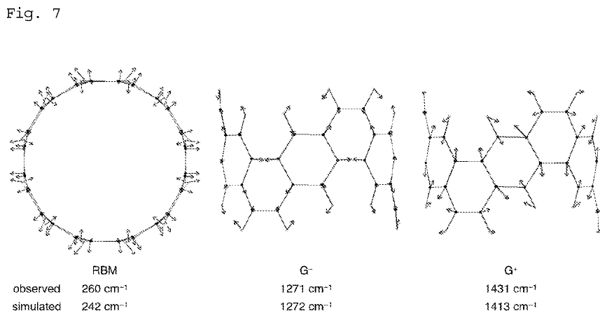

The Raman spectrum of [12]ICP was measured with a Via Reflex 48 (Renishaw) equipped with a 785-nm semiconductor laser. Raman signals were detected with a charge-coupled device (CCD). Laser beams were focused on a sample with an objective lens (magnification: 100×, numerical aperture: 0.85). The output of the laser was about 400 μW. The measurement was performed at room temperature under atmospheric conditions. FIGS. 6 and 7 illustrate the results.

1. A carbon nanobelt represented by formula (1): [symbol]
wherein each Ar is identical or different and represents an aromatic hydrocarbon ring; each R¹ is identical or different and represents hydrogen, alkyl, aryl, or alkoxy; and n represents an integer of 0 or more.

2. The carbon nanobelt according to claim 1 represented by formula (1A): [symbol]
wherein Ar and n are as defined above.

3. The carbon nanobelt according to claim 1, wherein Ar in formula (1) or formula (1A) represents a benzene ring or a naphthalene ring.

4. The carbon nanobelt according to claim 1, wherein n in formula (1) or formula (1A) represents an integer of 0 to 10.

5. A method for producing the carbon nanobelt of claim 1, the method comprising reacting, in the presence of a nickel catalyst, a cyclic compound represented by formula (2): [symbol]
wherein R¹ and n are as defined above; and each Ar′ is identical or different and represents an aromatic hydrocarbon ring having two halogen atoms as substituents.

6. The method according to claim 5, wherein Ar′ in formula (2) is a group represented by formula (5A) or (5B): 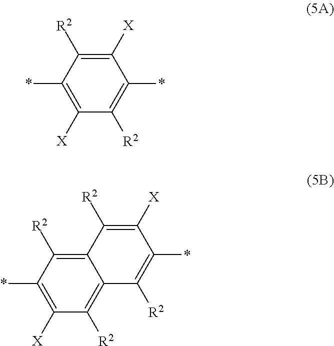
wherein each R² is identical or different and represents hydrogen, alkyl, aryl, or alkoxy; each X is identical or different and represents a halogen atom; and asterisk “*” represents a binding site.

7. The method according to claim 5, wherein the cyclic compound represented by formula (2) is obtained by reacting, in the presence of a base, a compound represented by formula (3A) or a salt thereof: 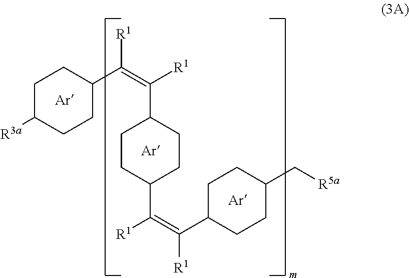
wherein Ar′ and R¹ are as defined above, R^{5a} represents triaryl phosphine or triaryl phosphite, m represents an integer of 0 or more, and R^{3a} represents acyl or a group represented by formula (4A), with the proviso that when m is 0, R^{3a} represents the group represented by formula (4A): 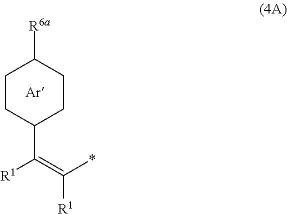
wherein Ar′ and R¹ are as defined above, and R^{ha} represents acyl.

8. A cyclic compound represented by formula (2): [symbol]
wherein each Ar′ is identical or different and represents an aromatic hydrocarbon ring having two halogen atoms as substituents; each R¹ is identical or different and represents hydrogen, alkyl, aryl, or alkoxy; and n represents an integer of 0 or more.

9. The cyclic compound according to claim 8, wherein Ar′ in formula (2) is a group represented by formula (5A) or (5B): 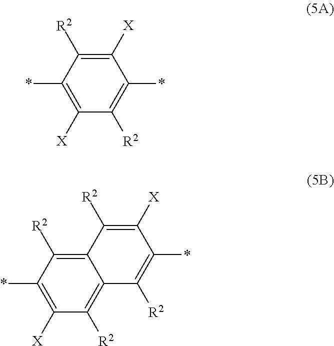
wherein each R² is identical or different and represents hydrogen, alkyl, aryl, or alkoxy; each X is identical or different and represents a halogen atom; and asterisk “*” represents a binding site.

10. A method for producing the cyclic compound of claim 8, the method comprising reacting, in the presence of a base, a compound represented by formula (3A) or a salt thereof: 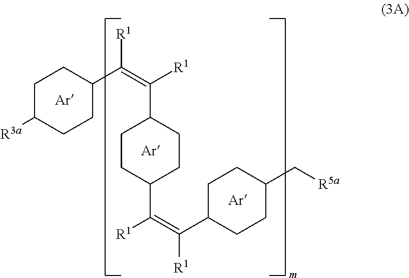
wherein Ar′ and R¹ are as defined above, R^{5a} represents triaryl phosphine or triaryl phosphite, m represents an integer of 0 or more, and R^{3a} represents acyl or a group represented by formula (4A), with the proviso that when m is 0, R^{3a} is the group represented by formula (4A): 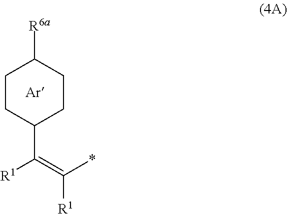
wherein Ar′ and R¹ are as defined above, and R^{6a} represents acyl.

11. A compound represented by formula (3) or a salt thereof: 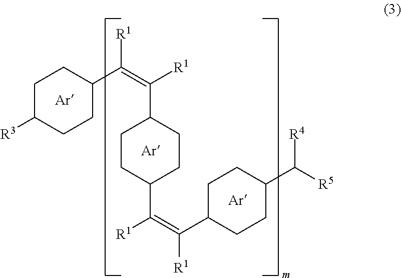
wherein each Ar′ is identical or different and represents an aromatic hydrocarbon ring having two halogen atoms as substituents; each R¹ is identical or different and represents hydrogen, alkyl, aryl, or alkoxy; R⁴ represents a hydrogen atom or a halogen atom; R⁵ represents a halogen atom, triaryl phosphine, or triaryl phosphite; m represents an integer of 0 or more; and R³ represents acyl, dialkoxymethyl, or a group represented by formula (4), with the proviso that when m is 0, R³ is the group represented by formula (4): 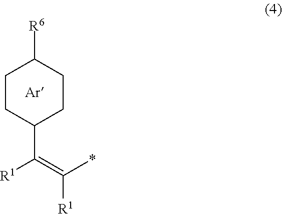
wherein Ar′ and R¹ are as defined above, and R⁶ represents acyl or dialkoxymethyl.
